(19) United States
(12) Patent Application Publication
(10) Pub. No.: US 2026/0325221 A1
(43) Pub. Date: Oct. 22, 2026

(54) Guiding the Insertion of a Surgical Needle With Inertial Measurements

(71) Applicants: Lohith J. KINI, San Francisco, CA (US); Brian HAAS, San Francisco, CA (US); Peter HADAR, Philadelphia, PA (US); The Regents of the University of California, Oakland, CA (US)

(72) Inventors: Lohith Kini, San Francisco, CA (US); Brian Haas, San Francisco, CA (US); Peter Hadar, Philadelphia, PA (US)

(21) Appl. No.: 19/490,275

(22) PCT Filed: Jun. 12, 2024
(86) PCT No.: PCT/US2024/033669

§ 371 Date: Dec. 4, 2025

Related U.S. Application Data
(60) Provisional application No. 63/521,167, filed on Jun. 15, 2023.

(51) Int. Cl. A61B 34/20; A61B 17/34

(52) U.S. Cl. CPC A61B 34/20; A61B 17/3403; A61B 2034/2048

(57) ABSTRACT

Provided are surgical devices for guiding the insertion of a needle along a desired three-dimensional trajectory from a surgical entry site to a surgical destination site. The device includes an element for securely attaching a needle to the housing of the device. The orientation of the device can be calibrated relative to the patient, and an inertial measurement unit (IMU) within the device allows the current orientation to be continuously monitored after calibration. The current orientation is compared to the desired orientation, and this relationship is transmitted to a display for viewing by the surgeon. As such, the surgeon is aware of whether the device and needle are oriented along the desired trajectory, thereby helping the surgeon to adjust the orientation and be confident the desired orientation is achieved. Methods of using such devices to perform the guided insertion of the needle are also described.

### CROSS-REFERENCE TO RELATED APPLICATION

This application claims priority to U.S. Provisional Patent Application 63,521,167, filed Jun. 15, 2023, the disclosure of which is incorporated by reference.

### INTRODUCTION

In 2020, 1.8 million Americans were diagnosed with cancer (1), and many underwent a biopsy for diagnosing and determining the stage of the cancer, constituting a 14 billion US Dollar market (2). Needle-based, image-guided biopsies and ablative therapies are important for diagnostic and therapeutic interventions with such cancer patients. For example, needles can be inserted to the site of the suspected cancer and a sample of the suspected tissue (i.e. a biopsy) can be collected through the needle. In other cases, a pharmaceutical drug can be delivered through the needle, such as a drug designed to inhibit the cancer. In other cases, the suspicious tissue can be ablated with either heat, cold, or microwave energy that is delivered via a probe inserted through the needle.

In many current procedures, the physician or proceduralist records an image of the biopsy or target site, such as with a computed tomography (CT) scanner. Afterwards, the proceduralist uses their spatial reasoning to estimate the approximate angle and depth of needle insertion. However, the natural human ability to estimate such angles and depths is poor compared to the needs of accurately reaching the suspected tissue. Such procedures can take large amounts of time and can be difficult for the proceduralist. As such, the ability to precisely target the lesion can be limited, thereby leading to unsuccessful procedures or false negatives due to under-sampling.

Xu et al. (US Patent Application Publication 2020/0197099) describes an instrument tracker that includes a case and an inertial measurement unit (IMU) inside the case. However, a surgeon using the Xu et al. device must apply continuous pressure to hold a surgical instrument against an outside surface of the instrument tracker. If such pressure is released or altered, then the instrument tracker can move or even fall. Hence, the Xu et al. procedure has the disadvantage of requiring continuous physical and mental attention from the surgeon.

### REFERENCES

(1) National Cancer Institute, “Cancer Statistics”, https://www.cancer.gov/about-cancer/understanding/statistics, retrieved 23 Mar. 2023.
(2) Research and Markets, “United States Biopsy Market, Volume & Forecast by Segments . . . Cancer, Companies”, October 2019, https://www.researchandmarkets.com/reports/4849351/united-states-biopsy-market-volume-and-forecast.

### SUMMARY

Provided are surgical devices for guiding the insertion of a needle along a desired three-dimensional trajectory from a surgical entry site to a surgical destination site. The device includes an element for securely attaching a needle to the housing of the device. The orientation of the device and needle can be calibrated relative to the patient, and an inertial measurement unit (IMU) within the device allows the current orientation to be continuously monitored after calibration. The current orientation is compared to the desired orientation, and this relationship is transmitted to a display for viewing by the surgeon. As such, the surgeon is aware of whether the device and needle are oriented along the desired trajectory, thereby helping the surgeon to adjust the orientation and be confident the desired orientation is achieved. Methods of using such devices to perform the guided insertion of the needle are also described.

### BRIEF DESCRIPTION OF THE DRAWINGS

FIG. 1A shows a perspective view of an exemplary surgical device.

FIG. 1B shows an alternate view of the FIG. 1A device.

FIG. 2 shows a schematic of a surgical device.

FIG. 3 shows a device with a needle inserted through the hole and an adjacent visual display.

FIG. 4 shows a device with a visual display that is mounted to the housing of the device, wherein the needle inserted therethrough is placed at a location on a surgical grid.

FIG. 5 shows an alternate view of the FIG. 4 device after it has been positioned as a desired pitch and yaw.

FIG. 6A shows an exploded view of a housing, an insert, and a needle.

FIG. 6B shows the insert being placed into the hole and rotated in order to lock it into position.

FIG. 6C shows the needle being inserted into the insert, which is located in the hole.

FIG. 7 shows a light and color sensor that is able to detect the color of the fluid as it passes through the center of the device.

FIG. 8 shows a device with a sensor on the bottom of its housing that emits a laser that can be used to detect the distance between the bottom of the device and the top surface of the body of the patient.

FIG. 9 shows different options for securing the needle in the hole, e.g. there can be a rubber gasket, a self-tightening twist lock, a magnetic, or an adhesive on the inner circumference to hole the needle in place.

FIG. 10 shows a spectroscopic sensor that is able to detect the chemical composition of a fluid as it goes through the needle and passes through the center of the device.

FIG. 11 shows a device with an EKG that assesses the patient.

FIG. 12 shows a flow sensor and the device.

FIG. 13 shows a pulse oximeter and a device.

FIG. 14 shows a respiration sensor.

FIG. 15 shows an ultrasound or doppler sensor.

FIG. 16 shows a second ultrasound or doppler sensor.

FIG. 17 shows a glucose monitor.

FIG. 18 shows a point of care diagnostics sensor.

FIG. 19 shows housings that have shapes other than a circular “donut” shape.

FIG. 20 shows an approximately cylindrical housing with “finger grip” sections that are indentations that can be used to receive the fingers of a surgeon, thereby increasing the dexterity of the device.

FIG. 21 shows additional shapes that can be used to increase dexterity and increase the ability of the surgeon's hand to easily control the orientation of the device.

FIG. 22 shows detachable shells, wherein the device can have the ability to mix and match the bottom and top parts of the shells.

FIG. 23A shows an embodiment of a housing with a hole, i.e. a through-hole.

FIG. 23B shows how a principle axis along the z-axis passes through the hole without intersecting the housing.

FIG. 23C shows how a reference plane in the x-y plane is perpendicular to the principle axis and is circumscribed by the housing.

FIG. 23D shows the volume consisting of contiguous hole points.

FIG. 24A shows an object with a blind hole.

FIG. 24B shows how axes entering the blind hole intersect the housing.

FIG. 25 shows an object with no indentations.

FIG. 26A shows an object with an indentation in the front wall.

FIG. 26B shows a principle axis passing through the indentation that does not intersect the object.

FIG. 26C shows indentation plane 265 that intersects principle axis 261 at point 264. Indentation plane 265 is not circumscribed by the object.

FIG. 27 shows the ability of the device and system to connect via BlueTooth, WiFi, NPC, and Android communications.

FIG. 28 shows the connection to an electronic medical record (EMR).

FIG. 29 shows the networking ability of the devices and systems, allowing them to communicate information and notifications to other devices, such as smartphones and pagers.

FIG. 30 shows that the devices and systems can integrate and communicate with a supply room software.

FIG. 31 shows an embodiment wherein the housing is attached to the skin of the patient with adhesive. As such, insertion of the needle to the surgical destination site is performed by moving the needle relative to the housing. The housing contain sensors, either optical or mechanical that measure the needle angular position and depth of insertion.

FIG. 32 shows an embodiment wherein the needle is in a fixed relationship with the housing, and the housing is not fixed to the skin. Thus, the needle is inserted by moving both the needle and the housing as a single unit, e.g. wherein a fastener retains the relative positions of both objects.

FIG. 33 shows an exploded view of a surgical system. The system includes a bottom section of the housing with electronic components placed thereon. Also shown s a top section of the housing with an “x” and “y” indicators of direction. Also shown is a display that gives can give the angel (e.g. in degrees) corresponding to the x and y directions. Also shown is a needle positioned with a spacer which has two flanges, e.g. so that it can be rotated and locked into position within the hole of the device.

FIG. 34 shows an exploded view of FIG. 33 wherein each individual component is shown separately.

FIG. 35A shows a perspective view of the assembled system of FIGS. 33 and 34.

FIG. 35B shows a side view of the FIG. 35A system.

FIG. 35C shows a bottom perspective view of the FIG. 35A system. Shown are three sets of pins that are electronic connectors, e.g. through a serial port.

FIG. 36A shows a top perspective view of a surgical device for guiding insertion of a needle along a fixed three-dimensional orientation, along with a needle.

FIG. 36B shows a side view of the FIG. 36A system.

FIG. 36C shows a bottom perspective view of the FIG. 36A system.

FIG. 36D shows an enlarged view of a section of FIG. 36C.

FIG. 36E shows an annotated view of FIG. 36D identifying the elements.

FIG. 36F shows an annotated view of FIG. 36D showing how the needle receiver can rotate perpendicular to the rails as well as translate along the length of the trails.

FIG. 36G is an annotated version of FIG. 36A that shows an approximation of roller-roller space 830.

FIG. 37 shows an exploded view of a surgical device for guiding insertion of a needle along a fixed three-dimensional orientation.

FIG. 38 shows an exploded the FIG. 37 device wherein all of the components are fully separated.

FIG. 39 shows an enlarged view of the rollers, needle receiver, and rails of FIG. 38.

FIG. 40A shows a top view of a device with a display and calibration buttons.

FIG. 40B shows a side view of the FIG. 40A device that includes finger receiving location 4002.

FIG. 40C shows the device and needle compared in size to a United States Quarter Dollar Coin.

FIG. 40D shows internal components of the device.

FIG. 40E shows internal components and the full size of the needle.

FIG. 41A shows a device with a concave out finger receiving location.

FIG. 41B shows a user positioning the device along a grid simulating a patient.

FIG. 41C shows an enlarged view of FIG. 41B.

FIG. 42 shows an embodiment the top of the device with a calibration button and display.

FIG. 43A shows a bottom perspective view of a pinching type bottom needle attachment element.

FIG. 43B shows the locations of pinching buttons and plungers.

FIG. 44 shows a device with a digital visual display having a target orientation and a current orientation shown by the visual display. The figure shows how the current orientation moves as the device is tilted towards the desired orientation.

FIG. 45 shows a device with a top circular display and a slanted finger receiving location.

### DETAILED DESCRIPTION

Provided are surgical devices for guiding the insertion of a needle along a desired three-dimensional trajectory from a surgical entry site to a surgical destination site. The device includes an element for securely attaching a needle to the housing of the device. The orientation of the device can be calibrated relative to the patient, and an inertial measurement unit (IMU) within the device allows the current orientation to be continuously monitored after calibration. The current orientation is compared to the desired orientation, and this relationship is transmitted to a display for viewing by the surgeon. As such, the surgeon is aware of whether the device and needle are oriented along the desired trajectory, thereby helping the surgeon to adjust the orientation and be confident the desired orientation is achieved. Methods of using such devices to perform the guided insertion of the needle are also described.

The housing of the device can have one or more finger receiving locations that help the hands of a user to securely and comfortable grip and manipulate the device and its attached needle. For example, there can be a second side surface that is curved and concave out, thereby providing a location for one or more fingers.

In some cases, the needle attachment element is a bottom needle attachment element (BNAE). This allows several advantages, including the ability to remove the surgical device from the top of the needle if it is no longer needed.

The BNAE can include a standardized shape that fits with most existing needles, such as a Luer slip and Luer lock shape. Hence, standard needles can be used with the surgical device without the needle for a special adapter. In other cases, the BNAE is a pinching BNAE that has a plunger or other element that securely pushes horizontally onto the needle, thereby helping to retain it.

In some cases, the needle attachment element is a hole in the device. By providing a hole that fully surrounds the needle, the surgical device can help maintain the needle in the desired three-dimensional orientation. In some cases, the housing adjacent to the hole provides friction against the needle that inhibits (e.g. prevents) the needle from moving, e.g. from sliding downwards due to gravity.

In some cases, the device includes a fastener that can provide friction against the needle, thereby inhibiting (e.g. preventing) the needle from moving, e.g. from sliding downwards due to gravity. For example, the fastener can be a “set screw” (e.g. as the term is used in American English). Such a set screw has threading on its exterior surface that matches the threading on the inside of an opening of the housing. Procedurally, the needle can be put into the desired position and then the set screw can be rotated, thereby causing one end of the set screw to contact the side of the needle. This contact generates a frictional force that inhibits further movement of the needle. In order to reposition the needle relative to the housing, the set screw can be rotated the opposite direction in order to remove the friction, and thereafter the needle can be moved, followed by the reapplication of friction from the set screw. In some cases, the fastener is a magnet, e.g. that can cause magnetic attraction to a metal needle, thereby inhibiting movement. In some cases, the magnet can be moved, thereby changing its magnetic force on the needle.

As such, the needle can be retained without the need for an operator to exert a continuous force against the needle. In some cases, the friction against the needle is not provided by the housing adjacent to the hole, but rather the friction is provided by a spacer that is inserted into the hole. Specifically, in such cases at least part of the spacer is inserted into the hole of the housing, and the spacer includes an opening that can receive a section of the needle. Thus, surfaces of the spacer adjacent to the opening provide friction that retains the needle at a fixed location relative to the device, so that they do not slip against one another, e.g. due to gravity. Moreover, spacers can be reversibly inserted and removed from the hole. As such, a single surgical device can have a single hole that can accommodate the insertion of different spacers that have different sized openings in order to accommodate different sized needles. Thus, systems are provided that include the surgical device along with two or more spacers, wherein each spacer can have a different sized opening to allow insertion of different sized needles while also applying sufficient friction to the needles to keep them from sliding downwards due to gravity. In some cases, the spacer is a gasket, e.g. a rubber gasket.

Systems are provided that include the surgical devices along with a display that visually indicates the relationship between a desired pitch and the current pitch of the device, along with the relationship between a desired yaw and the current yaw. Such displays can advantageously allow a surgeon to easily and continuously determine such angular relationships in real time. This ability is preferable to previous methods that required the uninformed spatial estimation of the surgeon, which can have significant human error. This ability is also preferable to previous methods that determined proper orientation through repeated CT scans or other medical images because such scans introduced complexity, difficulty, and sometimes radiation exposure.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a wire” includes a plurality of such wires. It is further noted that the claims may be drafted to exclude any element, e.g., any optional element. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Further, the dates of publication provided may be different from the actual publication dates which may need to be independently confirmed. To the extent the definition or usage of any term herein conflicts with a definition or usage of a term in an application or reference incorporated by reference herein, the instant application shall control.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### DEFINITIONS

The terms “hole”, “through hole” and “opening” are used interchangeably herein. A hole in the housing is a volume consisting of contiguous hole points (HPs) that are each independently located between two different points on the housing, wherein some of the HPs are located along a principle axis that does not intersect the housing, and wherein there is a reference plane that: (i) is perpendicular to the principle axis, (ii) includes some of the HPs, and (iii) is circumscribed by the housing. The statement that “the reference plane is circumscribed by the housing” can be understood by considering all the rays that begin at the intersection of the principle axis and the reference plane and which radiate outwards within the reference plane towards infinity. The reference plane is “circumscribed” by the housing if all of such rays intersect the housing. In contrast, if at least one of such rays does not intersect the housing, then the reference plane is not circumscribed by the housing.

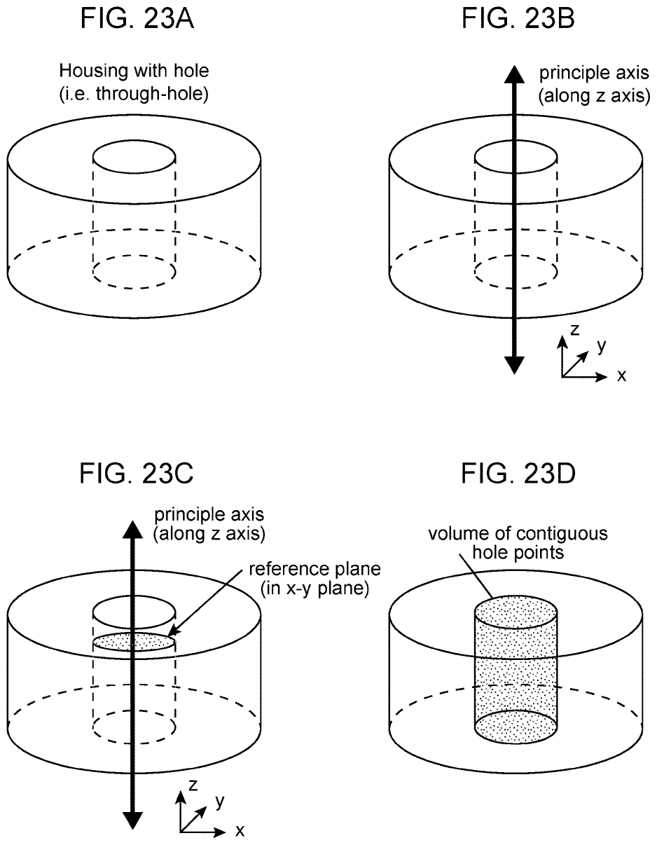

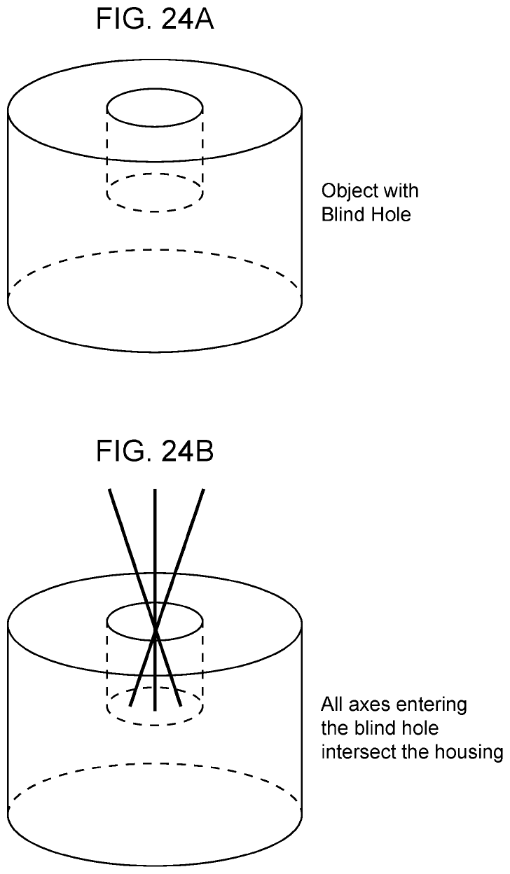

FIGS. 23A-D show an embodiment of a housing that illustrates the concept of a hole (i.e. a through-hole). FIG. 23A shows the housing with a short, cylindrical housing that includes a cylindrical hole. FIG. 23B shows a principle axis along the z axis that does not intersect the housing. Since the principle axis of FIG. 23B is along the z axis, the reference plane perpendicular to the principle axis will be an x-y plane, as shown in FIG. 23C. In particular, FIG. 23C shows a reference plane towards the top of the hole, wherein the housing circumscribes the reference plane. FIG. 24D shows the hole, i.e. the volume consisting of the contiguous hole points that are located between two different points on the housing.

A “blind hole” is a type of volume that is distinct from a “hole” or “through hole”. The term “blind hole” is used interchangeably herein with the term “cavity”. A blind hole in an object is a volume consisting of contiguous blind hole points (BHPs) that are each independently located between two different points on the object, wherein each axis passing through each BHP intersects with the object at one or more locations. Thus, whereas a hole (i.e. a through hole) has a principle axis that does not intersect the object, each axis containing BHPs in a blind hole does intersect the object.

FIGS. 24A-B show an embodiment of an object with a blind hole (i.e. a cavity). As shown in FIG. 24A, the cylindrical blind hole begins at the top of the object but stops before exiting the bottom of the object. FIG. 24B shows three exemplary axes that each enter the blind hole, but each of which intersect the housing.

An “indentation” in the housing is a volume consisting of contiguous indentation points (IPs) that are each independently located between two different points on the housing, wherein some of the IPs are located along an indentation axis that does not intersect the housing, and wherein there are indentation planes that are perpendicular to the indentation axis and that each include some of the IPs, wherein none of the indentation planes are circumscribed by the housing.

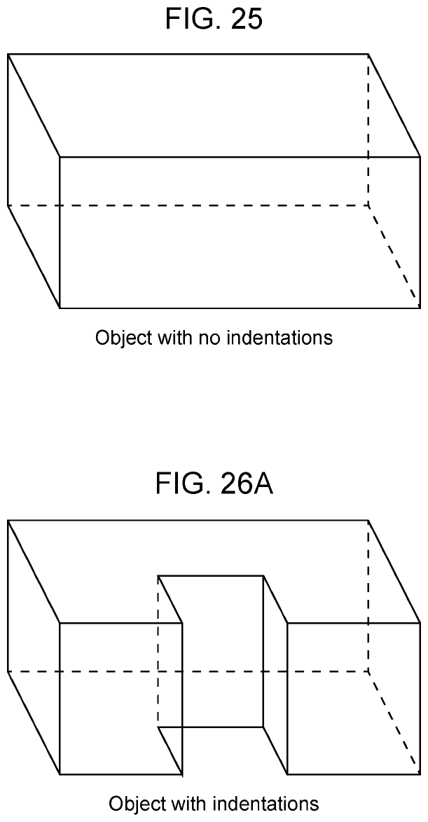

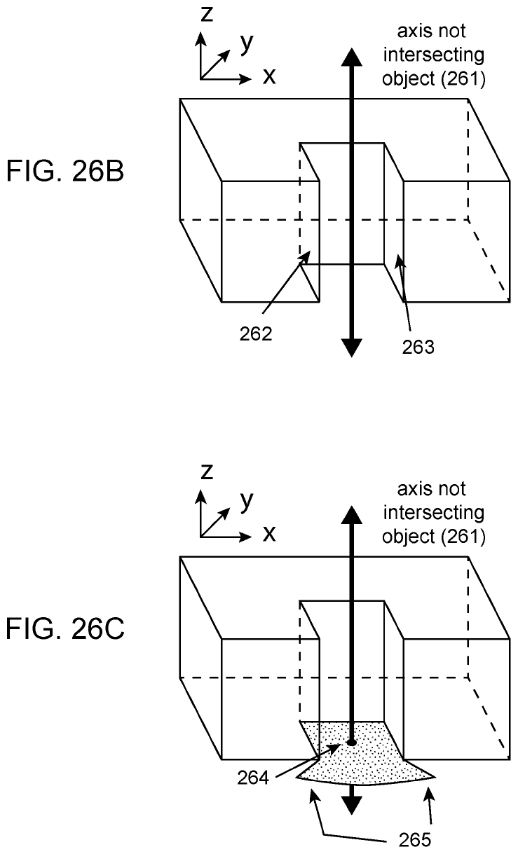

FIG. 25 shows a reference object that has no indentations. FIGS. 26A-C show an object with an indentation. FIG. 26A shows an object wherein there is a rectangular indentation in the front wall. FIG. 26B shows a principle axis along the z-axis that does not intersect the object, wherein the principle axis is located between side 262 and side 263. FIG. 26C shows indentation plane 265 that is located in the x-y plane and is perpendicular to the principle axis that is in the z-direction. Indentation plane 265 meets principle axis 261 at point 264. As shown in FIG. 26C, a section of indentation plane 265 radiating from point 264 intersections the object. However, some of the rays originating at point 264 and radiating outwards in indentation plane 265 do not intersect the object. Therefore, indentation plane 265 is not circumscribed by the object. Since no plane perpendicular to principle axis 261 is circumscribed by the object, then the region is referred to as an indentation.

The terms “user”, “surgeon”, and “physician” are used interchangeably herein.

### Devices With Needle Attachment Element

Provided are surgical devices for insertion of a needle along a desired three-dimensional trajectory from a surgical entry site to a surgical destination site. In some embodiments, the device includes:
a housing;
a needle attachment element;
a spatial orientation indicator on an external surface of the housing;
an inertial measurement unit (IMU) inside the housing; and
an electronic communication element.

As used herein, a “needle attachment element” is a structure that causes the needle to remain in a fixed spatial relationship with the housing despite gravitational forces, such as the gravitational force at the surface of the earth of 9.8m/s 2 . Stated in another manner, once a needle is attached to the housing with the needle attachment element, such gravitational forces are insufficient to cause the needle to move relative to the housing. Therefore, the needle will remain in the fixed spatial relationship with the housing even if a surgeon is not actively trying to keep the needle and housing together. Hence, the needle attachment element beneficially frees the hands and minds of the surgeon to perform other tasks.

The needle attachment element causes this fixed spatial relationship by exerting forces on the needle or blocking certain motions. The needle attachment element can also help prevent accidental movements due to accidentally bumping the device or needle. Since the needle is typically inserted into the body of a patient, the attachment element can also help retain a fixed spatial relationship despite external forces from the body that push on the needle.

In a first embodiment, the needle attachment element is a bottom needle attachment element that is located on a bottom surface of the housing. For instance, the element can be a Luer lock element with threading, and the needle can be inserted and twisted into the Luer lock, thereby securely attaching the needle to the housing. The element can also be a Luer slip element with a conical section, wherein the needle can be pushed onto the conical section, thereby providing friction and attachment. In some cases, the needle is inserted into a cavity of the bottom needle attachment element that has a spring-loaded plunger that engages with the needle, thereby securing it. In such cases, the device can be referred to as including:
a housing;
a bottom needle attachment element located at a bottom surface of the housing;
a spatial orientation indicator located on an external surface of the housing;
an inertial measurement unit (IMU) inside the housing; and
an electronic communication element.

In a second embodiment, for example, the needle attachment element comprises an inner surface of a hole in the housing, wherein the needle can be inserted through the hole and friction between the needle and the inner surface retains the relative spatial relationships. In such embodiments, the housing comprises a hole. In such embodiments, the surgical device can be referred to as comprising:
a housing;
a needle attachment element comprising an inner surface of a hole in the housing;
a spatial orientation indicator on an external surface of the housing;
an inertial measurement unit (IMU) inside the housing; and
an electronic communication element.

### Devices With a Bottom Needle Attachment Element

As discussed above, in some cases the needle attachment element is a bottom needle attachment element (BNAE) that is located on a bottom surface of the housing. For instance, the bottom needle attachment element can be connected to the bottom surface of the housing with glue, a screw, or a bolt.

In some cases, the BNAE comprises a conical exterior surface. For instance, in the medical field needles are commonly attached to syringes through a “Luer slip” connector on the syringe. This Luer slip connector has a conical exterior surface and a lumen that passes through the middle of the Luer slip connector to allow liquid to flow through the needle and into the barrel of the syringe. This Luer slip tip can be used as the BNAE for the current devices since pushing the Leur slip tip and the needle together will wedge the parts together and they will remain together due to friction. Luer slip connectors are described by International Standards Organization (ISO) 80369-7:2021, and in some cases the BNAE has exterior dimensions that are within the scope of this ISO standard. Optionally, the BNAE can omit the liquid passage since the purpose is to connect the needle to the housing, and not to transfer liquids.

In some cases, the BNAE comprise a cylindrical section and internal screw threads on an internal surface of the cylindrical section. For example, the BNAE can employ a “Luer lock”, e.g. as described by ISO 80369-7:2021. This Luer lock has a cylindrical section with internal screw threads on an internal surface of the cylindrical section, i.e. that correspond to external screw threads on the needle. Therefore, screwing the needle's threads into the threads of the Luer lock cylinder allows the generation of friction between the two sets of screw threads, thereby helping to keep the needle attached to the housing.

In some cases, the BNAE is a spring plunger BNAE comprising:
a body comprising a flange;
a cavity in the body;
a plunger; and
a spring,
wherein the spring is positioned between the body and the plunger,
wherein a first section of the plunger is positioned between the spring and the cavity,
wherein a second section of the plunger is positioned between the spring and the flange.

This arrangement of elements allows the plunger to reversibly move inwards into the cavity or outwards towards the body. Thus, in its resting state, the spring exerts a force that pushes the plunger inwards, but the flange prevents the plunger from moving too far inwards. When the needle is inserted into the cavity, the needle will have a needle flange that presses the plunger outwards and against the spring, thereby allowing the needle to be inserted. Once the needle flange passes the plunger, the spring will push the plunger inwards. Thus, the plunger will be located behind the needle flange, thereby helping to keep the needle fully inserted. Spring plungers are known in field of mechanical engineering and are commercially sold by companies such as McMaster-Carr and Halder USA. The plunger can have any suitable shape, such as a sphere or a cylinder. In some cases, the needle can be removed from the spring plunger BNAE simply by exerting a sufficient outwards force. In other cases, a second mechanical button is depressed that unlocks the needle from the plunger.

In some cases, the BNAE is a pinching BNAE comprising:
a body;
a cavity in the body;
a pinching element; and
a rotatable object that is partially located at an outer surface of the body,
wherein the pinching element is positioned between the body and the cavity,
wherein rotating the rotatable object causes the pinching element to move inwards towards the cavity.

In an “open” state, the pinching element can be mostly or fully retracted into the body of the BNAE. Thus, the cavity is mostly or fully open, thereby allowing insertion of a needle into the cavity. Afterwards, the rotatable object can be rotated in order to cause the pinching element to move inwards into the cavity and change to the “closed” state. For example, the rotatable object can be a wheel or knob partially located at an external surface of the body of the BNAE. As such, the pinching element can apply pressure to the needle, thereby holding it in place through friction. The pinching element can also move inwards behinds a flange of the needle, i.e. wherein trying to remove the needle will cause the needle flange to contact the pinching element, thereby preventing removal of the needle from the cavity.

### Devices With a Hole in the Housing

In some cases, there is a hole in the housing of the device.

In some cases the needle attachment element comprises an inner surface of a hole in the housing, wherein the needle can be inserted through the hole and friction between the needle and the inner surface retains the relative spatial relationship.

The hole in the housing is dimensioned such that a needle can be inserted through the hole. The section of the housing that is adjacent to the hole is referred to as the “inner surface of the housing”.

For example, in some cases the needle ranges from a 34 gauge needle to a 6 gauge needle In some cases, the needle gauge ranges from 34 gauge to 10 gauge. In some cases, the outer diameter of the needle ranges from 0.159 mm to 3.404 mm. In some cases, the hole has a circular cross-section. As such, in cases wherein the needle is inserted directly through the hole, the inner diameter of the hole can be slightly larger than the outer diameter of the needle in order to allow insertion of the needle into the hole. Thus, in some cases the hole has an inner diameter ranging from 0.17 mm to 4.0 mm. In some cases, the hole has an inner diameter than is larger than the outer diameter of the needle by from 1% to 25%. In some cases, the geometric center of the device is located in the hole, wherein the geometric center of the device is the arithmetic mean of all points on the housing of the device. In some cases the hole has a circular cross-section, e.g. wherein the circular cross-section is perpendicular to a principle axis that does not intersect the housing.

As discussed above, in some cases the needle is inserted directly into the hole, i.e. such that there is physical contact between the needle and a section of the housing adjacent to the hole.

In other cases, a spacer comprising an opening is placed such that some or all of the spacer is located inside the hole. As such, the needle can be inserted into the opening of the spacer and therefore the needle is also inserted into the hole of the housing. The insertion of the needle into the opening means that a section of the needle is located within the opening. When the needle is inserted into the opening then there is physical contact between a section of the needle and the spacer.

As used herein, the term “spacer” is used interchangeably with the terms “shim” and “insert”. As discussed herein, the spacer is not considered a part of the surgical device. Instead, the present disclosure provides systems that include the surgical device and a spacer comprising an opening, wherein the spacer has outer dimensions allowing a section of the spacer to be inserted into the hole of the housing. For example, in some cases the hole has a circular cross-section and an inner diameter that is larger than the outer diameter of a circular cross-section of the spacer.

In some cases, the opening of the spacer has a circular cross-section. The opening in the spacer can have any suitable inner dimension, e.g. an inner diameter ranging from 0.17 mm to 4.0 mm. In cases wherein the spacer is used, the hole in the housing can be significantly larger than the diameter of a needle being used. By having a larger hole, a single device can accommodate different spacers that can be specifically selected to accommodate specific sizes of needles. As such, in some cases the system includes a first spacer and a second spacer, wherein the first and second spacers have openings with different sized diameters, e.g. to accommodate different sized needles. In some cases, the spacer is retained in the hole by friction between the spacer and the housing. In some cases, the spacer can be rotated to “lock” the spacer to the housing and rotated in an opposite direction to “unlock” it from the housing. In some cases, the device includes two or more clamps that can be rotated. This rotation can cause the size of the hole in the device to decrease, thereby causing the housing can contact and apply pressure to the spacer, thereby retaining the spacer in the hole. In some cases, the clamps can be adjusted independently of each other, e.g. such that the center of the opening of the spacer is at the center of the hole of the housing.

The housing can have a variety of exterior dimensions and shapes. For instance, in some cases the housing has a circular cross-section that is perpendicular to the principle axis of the hole. The housing can have a flat bottom surface in some embodiments, e.g. so the device can rest securely on the skin of the subject. In some cases the housing has a flat top surface. In some embodiments, the external dimension of the housing has the shape of a cylinder with either straight edges or rounded edges, e.g. wherein the hole for insertion of the needle connects the top circular surface to the bottom circular surface. In some cases the diameter of the cylinder is greater than its height. In some cases the diameter of the cylinder is equal to or less than its height.

The shape of the housing can also be described by the term “genus”, which is a term from the field of mathematical topology. As used herein, the “genus” of a connected, orientable surface is an integer representing the maximum number of cuttings along non-intersecting closed simple curves without rendering the resultant manifold disconnected. For example, the term genus is discussed by Munkres (Topology. Vol. 2. Upper Saddle River: Prentice Hall, 2000). The housing of the surgical device will have a genus of 1 or more, such as 1, 2, or 3. In some cases, the housing has a genus of 1.

In some cases, there is a hole in the housing and the needle attachment element is a needle mount attached to the housing. In such embodiments, the needle attachment element is not an inner surface of the housing, but instead it is a separate structure (i.e. the needle mount) that attaches the needle. For example, the needle mount can comprise three screws that penetrate the inner surface of the housing and go into the hole. As such, rotating the three screws can move them a variable distance into the hole. The screws can contact the needle. As such, by changing the relative position of the three screws, the angle of the needle can be adjusted. Such screws are sometimes referred to as “set screws” in American English. Any suitable number of such screw can be used, such as two, three, four, five, six, or more.

### Finger Receiving Location

The housing can include a “finger receiving location” (FRL) that is dimensioned to accommodate certain fingers of a human hand comfortably and securely. This can beneficially increase the comfort and ease of using and moving the device.

The finger receiving location can be best understood by first considering the housing in general. In some cases, the housing can include a top surface, first side surface, and a bottom surface. In some cases, the top surface has a largest dimension (e.g. diameter) ranging from 1 cm to 8 cm. In some cases the first side surface has a height ranging from 1 cm to 8 cm. In some cases, the top surface and bottom surface are parallel to each other. In some cases, the first side surface is perpendicular to the top surface, bottom surface, or both. In some cases, the top surface is circular, square, or rectangular. In some cases, the first side surface has a horizontal cross section that is circular, square, or rectangular.

In some embodiments, the finger receiving location is an “undercarriage” FRL that is a second surface that:
is connected to the first side surface along the entire bottom perimeter of the first side surface,
is connected to the bottom surface along the entire perimeter of the bottom surface, and has a larger horizontal cross section at its top than at its bottom.

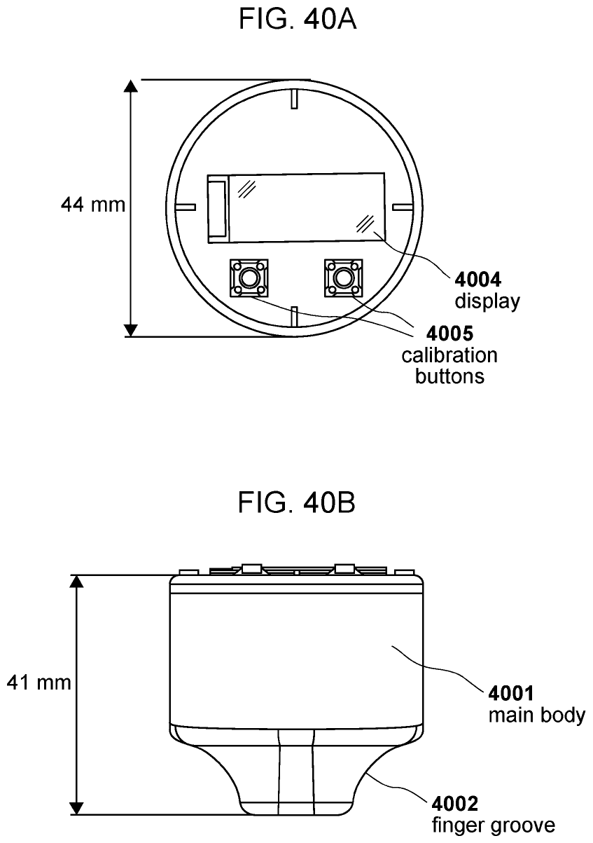

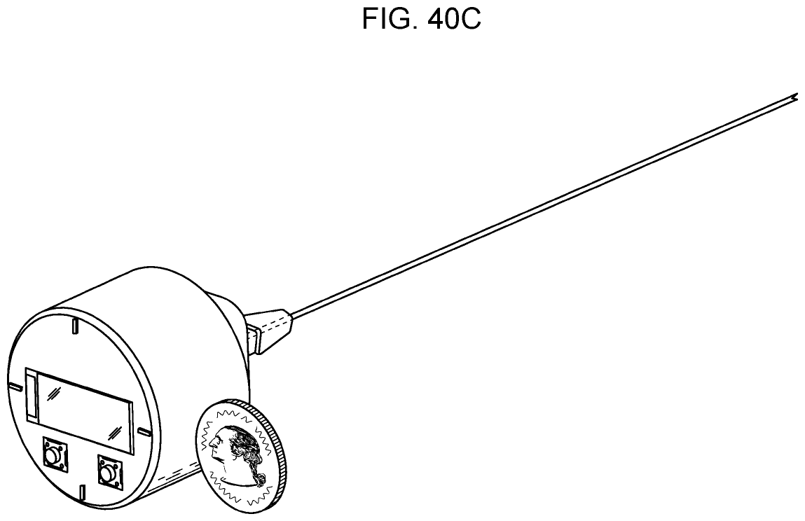

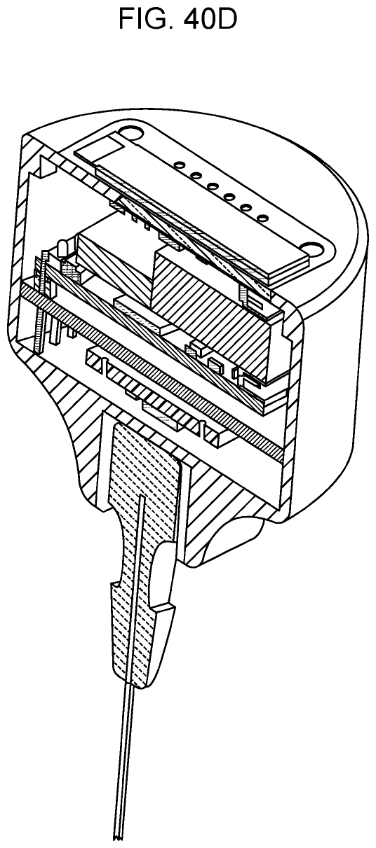

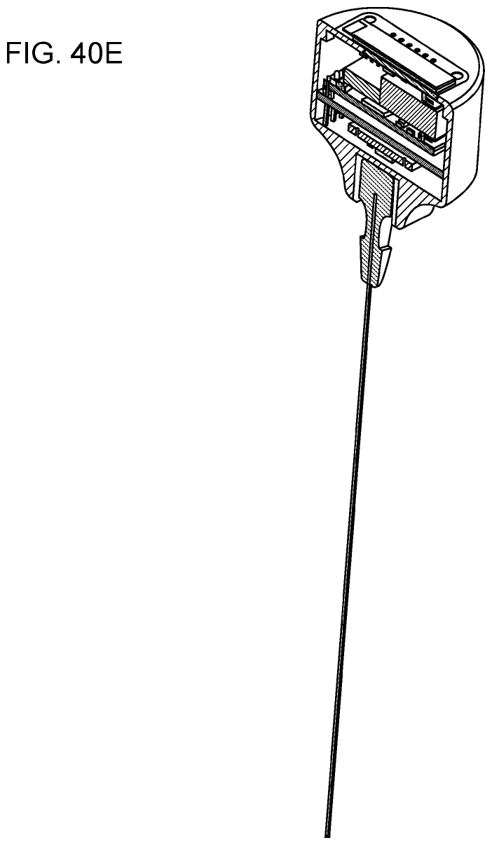

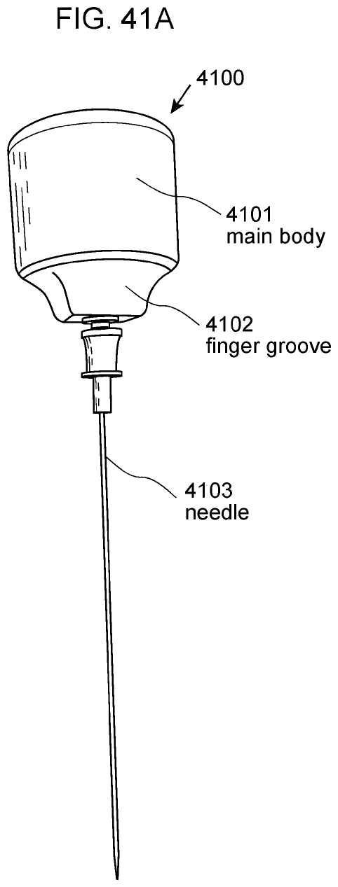

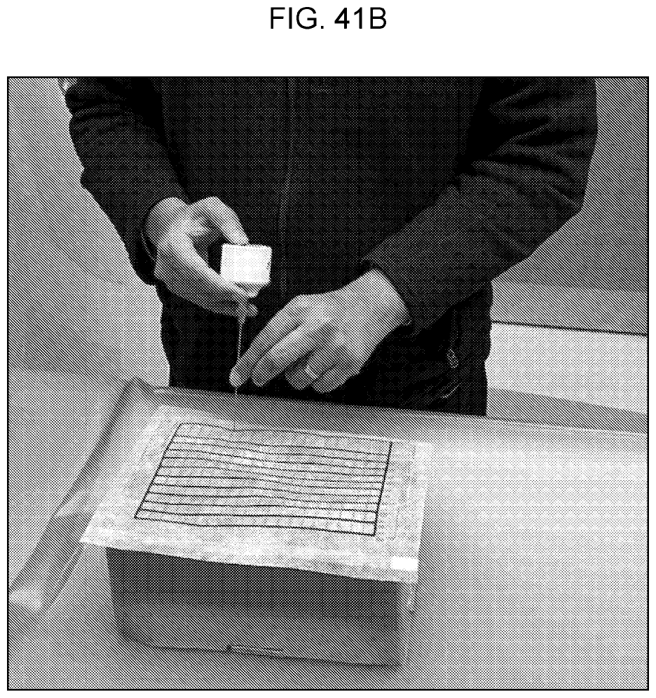

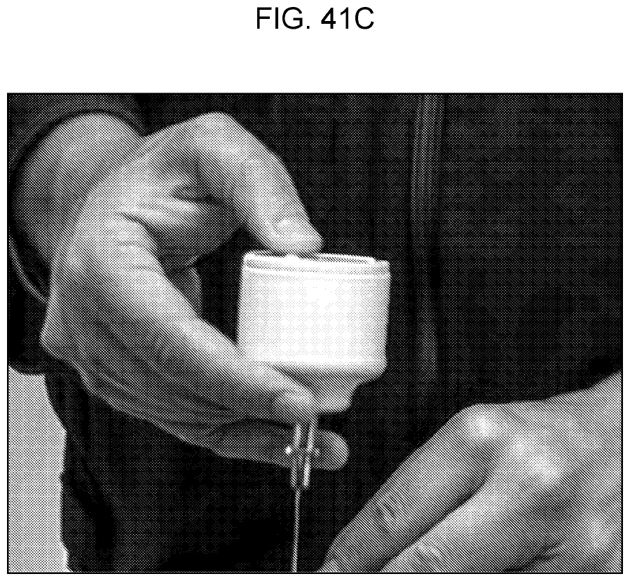

An example of an undercarriage FRL is shown in FIGS. 40B and 41A-C. In this embodiment, the top surface is a flat circle and the first side surface is the vertical, cylindrical section. At the index finger of the person, the first side surface is connected to the second side surface, which decreases in diameter while moving downwards. The connection between the first and second side surfaces is along the entire bottom perimeter of the side surface, i.e. since both surfaces wrap 360° around the device. The second side surface also connects to the enter perimeter of the bottom surface, which is horizontal. Behind the index finger of the user is the bottom needle attachment element that retains the needle.

In some cases, the second side surface FRL is curved, such as concave out. In some cases, the second side surface has a height ranging from 5 mm to 40 mm. In some cases, both the first and second side surfaces each comprise circular cross sections. In some embodiments these cross sections are coaxial, i.e. the centers of both circular cross sections are located along an axis that is perpendicular to both cross sections. In some cases, the second side surface has circular cross sections that decrease in diameter when moving from the top of the second side surface to the bottom of the second side surface.

In some embodiments, the finger receiving location is a “slanted” FRL that is a second surface that:
is connected to the first side surface along a part of the perimeter of the first side surface,
is connected to the bottom surface along a part of the perimeter of the bottom surface, and
has a larger horizontal cross section at its top than at its bottom.

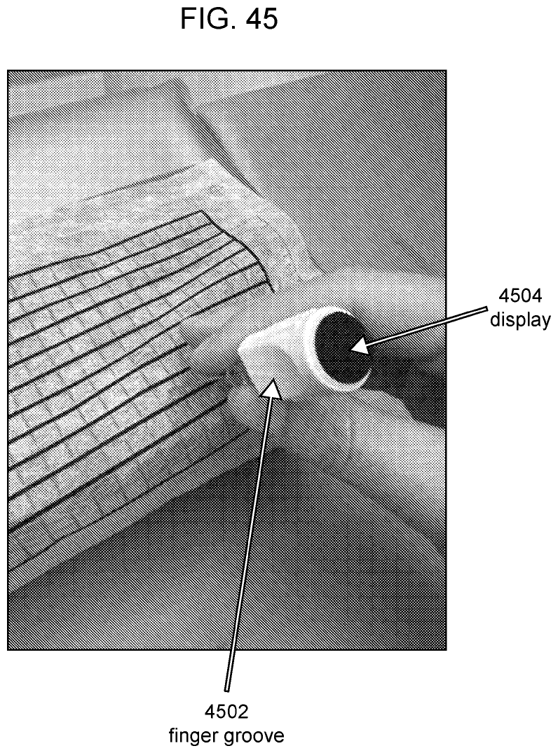

An example of a slanted FRL is shown in FIG. 45. Whereas the undercarriage FRL side surface connected to the entire perimeters of the first side surface and bottom surface, in a slanted FRL the second side surface only connects to a portion of such surfaces. Stated in another manner, the undercarriage FRL wraps 360° and completely around the device, whereas the slanted FRL does not completely wrap around the device.

In some embodiments of the slanted FRL, the second side surface is planar. For example, the slanted FRL could be viewed as cutting an angled plane into the first side surface.

In other cases, the second side surface in the slant FRL is curved and concave out. As such, the slanted FRL can be considered to have a greater depth towards its center than at its edge, thereby providing for a more secure fit of the human finger.

In some cases, there are multiple slanted FRLs, such as a second side surface and a third side surface that it is on the opposite side of the housing from the second side surface. For example, the human thumb can be placed in the second side surface and the human index finger can be placed in the third side surface. As such, the thumb and index fingers can be pushed together in order to securely hold the device.

### Additional Aspects of the Devices

In some cases, the housing comprises one or more exterior indentations. This indentation in the housing can be dimensioned such that part of a finger of a user (e.g. a surgeon) can be placed within the indentation. As such, the user can more easily and accurately control the position and orientation of the device by exerting forces at the section of the housing adjacent to the indentation. For example, the indentation can have a semi-circular cross-section and a diameter of about 1 cm to 2 cm, and therefore the indentation is dimensioned to receive the fingertip of a user. In some cases, the indentation has a depth ranging from 5 mm to 4 cm. In some cases, the indentation has a width or a diameter ranging from 5 mm to 4 cm. In some cases, the housing comprises two, three, or four exterior indentations that each independently have a depth ranging from 5 mm to 4 cm, e.g. to accommodate two, three, or four fingers of a surgeon.

In some cases, the exterior indentation in the housing is dimensioned such that multiple fingers of a user can be accommodated within indentation. For example, the indentation can have a depth ranging from 5 mm to 10 cm. In some cases, the indentation has a width or length ranging from 4 cm to 15 cm. In some cases, the indentation has a length or width that is greater than 15 cm, e.g. wherein the entire housing itself is shaped to make gripping the housing easier. For example, the housing can have an hourglass shape, i.e. a middle section with a smaller diameter and two end sections on opposite sides of the middle section that have larger diameters than the middle section.

As described above, the surgical device includes a spatial orientation indicator on an external surface of the housing. The spatial orientation indicator can be used to calibrate the initial three-dimensional orientation of the device relative to a known object. After such calibration, the inertial measurement unit can record changes in the inertia of the surgical device, thereby allowing a computer to monitor the current three-dimensional orientation of the device. In some cases, the calibration of the initial orientation involves a gravitational vector that is measured by the inertial measurement unit.

In some cases the spatial orientation indicator is a marking, e.g. a marking having a different color than the adjacent sections of the external surface of the housing. In some cases, the spatial orientation indicator includes two perpendicular line segments. In some cases, the spatial orientation indicator includes a protrusion that protrudes from adjacent sections of the external surface of the housing. For example, the spatial orientation indicator can be dimensioned for alignment with a grid of lights from a computer tomography (CT) scanner or a magnetic resonance imaging (MRI) scanner. For instance, Wu et al (Journal of Applied Clinical Medical Physics, 2014, 15, 2, 138, doi: 10.1120/jacmp.v15i2.4544) and Mackey (US Patent Application Publication 2007/0269016) describe physical structures and methods for calibrating the orientation of an object relative to a radiological scanner, such as a CT scanner. In some cases, the spatial orientation indicator comprises two or more protrusions. In some cases, the spatial orientation indicator comprises two or more markings.

The surgical device also includes an inertial measurement unit (IMU) located inside the housing. The IMU includes one or more inertia sensors, such as two or more, three or more, four or more, or five or more. In some cases one or more of the inertia sensors is a gyroscopes, e.g. a microelectromechanical system (MEMS) gyroscope or a Coriolis vibratory gyroscope (CVG). In some cases, one or more of the inertia sensors is an accelerometer. In some cases, one or more of the inertia sensors is a magnetometer. Ahmad et al is a review discussing various types of inertia sensors and IMUs that can be used in the present surgical devices (International Journal of Signal Processing Systems, 2013, 1, 2, doi: 10.12720/ijsps.1.2.256-262).

The IMU generates inertial measurement data (IMD) based on changes in inertia, which relate to changes in the position and three-dimensional orientation of the IMU and the device. If the IMU generates data from a gyroscope, then such data is referred to a gyroscopic data. If the IMU generates data from an accelerometer, then such data is referred to as accelerometer data. The IMU to transmit inertial measurement data at any suitable frequency. In some cases, the time interval between transmissions of inertial measurement data ranges from 0.01 ms to 1 second, such as from 0.1 ms to 500 ms or from 1 ms to 100 ms.

The surgical device also includes an electronic communication element. This electronic communication element electronically transmits the inertial measurement data from the inertial measurement unit (IMU) a controller. In some cases, the controller is also located within the housing of the surgical device. In such cases, the electronic communication element can simply be an electronic communication cable, such as a USB cable. In other cases, the controller is located outside the housing. In some embodiments, the electronic communication element can be an electronic communication cable (e.g. a USB cable) that exits the housing and connects to the controller. In other embodiments, the electronic communication element can include an electronic communication cable and a wireless electronic transmitter, such as a Bluetooth® or Wi-Fi transmission element. As such, the inertial measurement data can be transmitted from the IMU through the cable to the wireless electronic transmitter, which can then transmit the data wirelessly to the controller. As such, the controller receives the inertial measurement data from the electronic communication element.

Chronologically, the controller first receives information about the desired trajectory from the surgical entry site to the surgical destination site. For example, the entry site can be a particular section of abdominal skin and the destination site can be a section of the pancreas that appears to have a cancerous tumor. Additionally, a particular coordinate system is selected, such as the axes of a computer tomography (CT) scanner. Based on this coordinate system, the entry site, and the destination site, the controller will know the particular angles required to move the distal end of the needle from the entry site to the destination. Such angles can be expressed as the pitch and yaw of the desired trajectory, e.g. wherein the movement of an airplane flying through the air can also be described based on its pitch and yaw.

Next, the three-dimensional orientation of the surgical device relative to the reference axes can be determined. For example, the spatial orientation indicator on the external surface of the housing can be aligned with alignment laser lights from the CT scanner. Electronic instructions can be sent to the controller stating that the surgical device has a particular orientation relative to the reference axes of the CT scanner. In some cases, the system includes a distance sensor, e.g. a laser range finder. In some cases, the distance sensor is attached to a bottom surface of the housing, thereby allowing measurement between the bottom of the housing and the skin of the patient. In some cases, the determination of the spatial orientation is performed based on both the IMU data and the distance sensor data (DSD).

After determination of the initial orientation of the surgical device, the IMU can continually record and send inertial measurement data to the electronic communication element and the controller. As such, the controller can use each sequential set of inertial measurement data to determine the updated, current orientation of the surgical device. Therefore, the initial calibration of the surgical device and the continuous recording of inertial data allows for the current pitch and yaw of the surgical device to be known.

Additionally, the controller compares the current orientation of the device to the desired orientation based on the desired trajectory. The current and desired orientations can be expressed as pitch and yaw. As such, the electronic communication element that electronically transmits inertial measurement data (IMD) from the IMU to a controller configured to determine the relationships between the device's current pitch and yaw and the pitch and yaw of the desired trajectory by comparing an initial yaw, an initial pitch, the IMD, and the desired three-dimensional trajectory.

In some cases, the surgical device includes two directional markings that are located on an exterior of the housing and each have a different color than surrounding regions of the exterior of the housing. Such color differences making the markings visually recognizable to a surgeon or other user. For example, the directional markings can be black and the surrounding regions of the housing can be white. The directional markings correspond to the pitch and yaw of the device, thereby helping the surgeon remember how to move the device to achieve a desired pitch and yaw.

For example, in some cases the first marking comprises the word “pitch” and the second marking comprises the word “yaw”. In other cases the markings comprise the words altitude and azimuth, the letters x and y, the letter x and z, the letters y and z. In some cases, the markings comprise the Greek letters theta and phi. In some cases, each of the two directional markings comprise an arrow, i.e. indicating the spatial direction corresponding to each word or letter. In some cases, the symbol (e.g. word or letter) of the directional markings is also shown on a corresponding region of the display. For instance, the directional markings can have the letters “x” and “y” and the display shows the letters “x” and “y” adjacent to the numerical relationships between the pitch and yaw.

In some cases, one or more components of the devices and the systems are connected to a battery. In some cases, one or more components of the devices and systems are connected to a “domestic electricity supply”, which can also be referred to as “mains electricity”.

### Device for Insertion Along a Fixed Three-Dimensional Orientation

As discussed above, the present disclosure provides surgical devices and systems for insertion of a needle along a desired three-dimensional trajectory. Such devices employ an inertial measurement unit (IMU) to calibrate and continually determine the current three-dimensional orientation of the housing of the device, and therefore the needle passing through the opening in the housing.

However, the present disclosure also provides for a distinct type of device which can also be used for guided surgical insertion of a needle. This second type of device does not require an inertial measurement unit (IMU).

This second type of surgical device includes:
a base comprising a base hole;
an upper housing comprising an upper hole;
a track comprising a first rail and a second rail;
a needle receiver comprising a needle hole; and
a needle aligner comprising a first roller and a second roller.

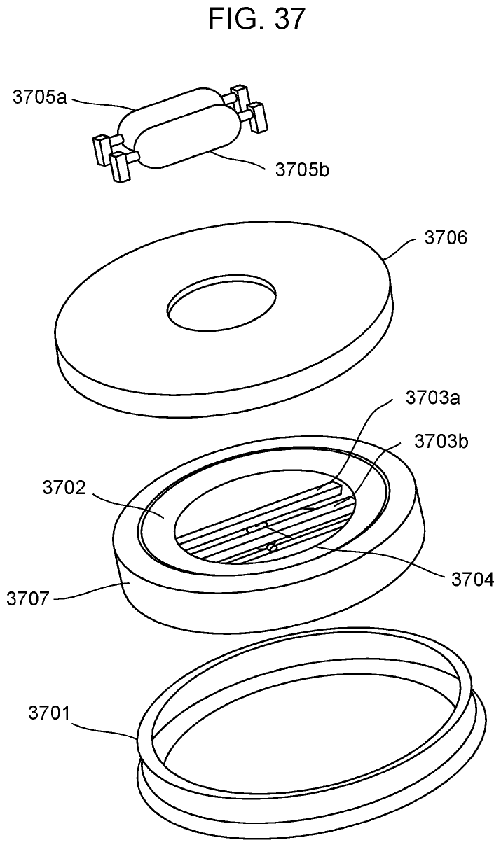

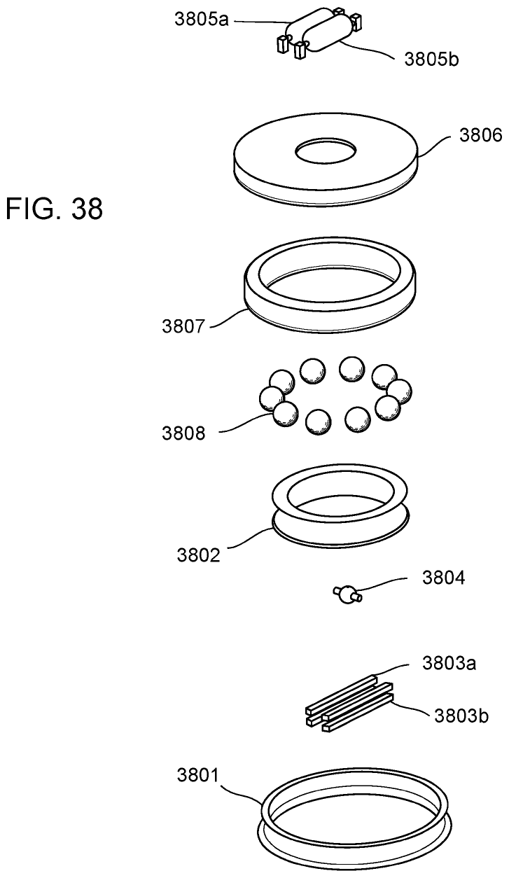

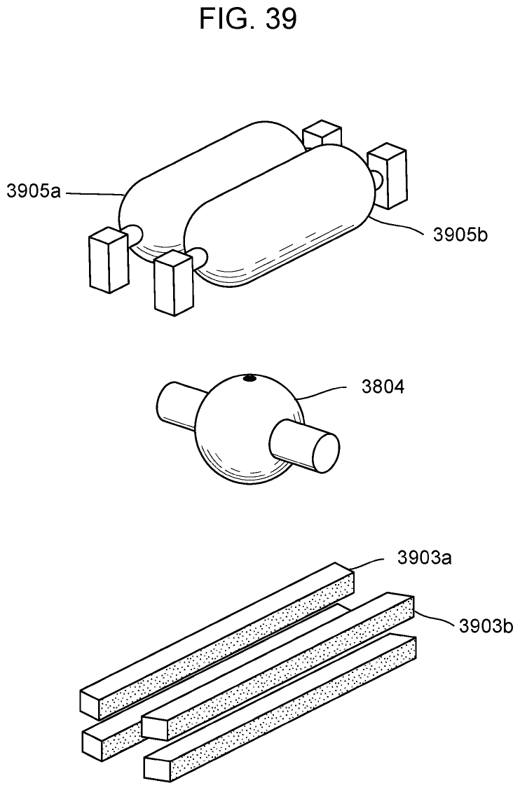

For example, FIG. 37 shows an exemplary device 800 that includes base 801, upper housing 802, track 803 with first rail 803a and second rail 803b, needle receiver 804, and needle aligner 805 comprising first roller 805a and second roller 805b. Also shown are optional element 806, which is a cover plate. Furthermore, FIGS. 37 and 38 show optional middle housing 807 and optional ball bearings 808.

The device can be described relative to a principle axis that extends vertically, a first horizontal axis extending left and right, and a second horizontal axis extending backwards and forwards.

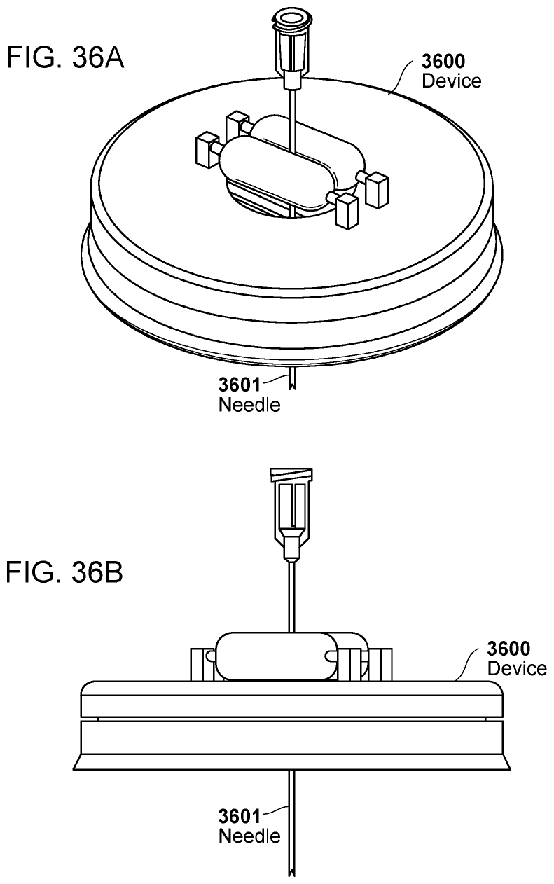

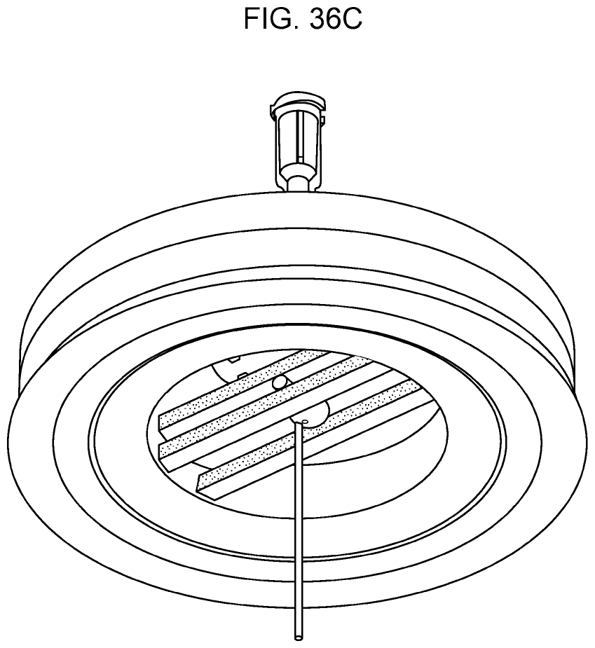

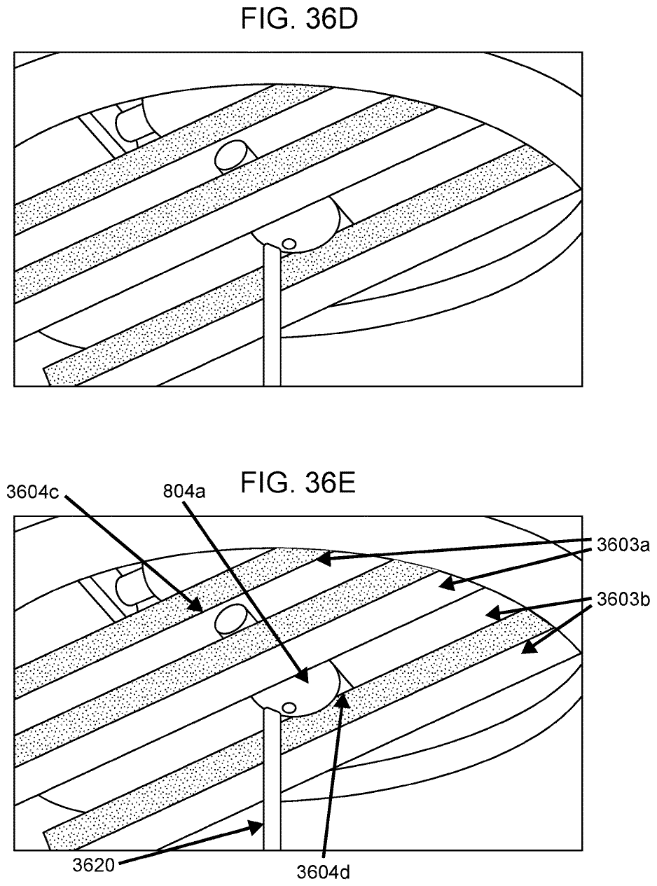

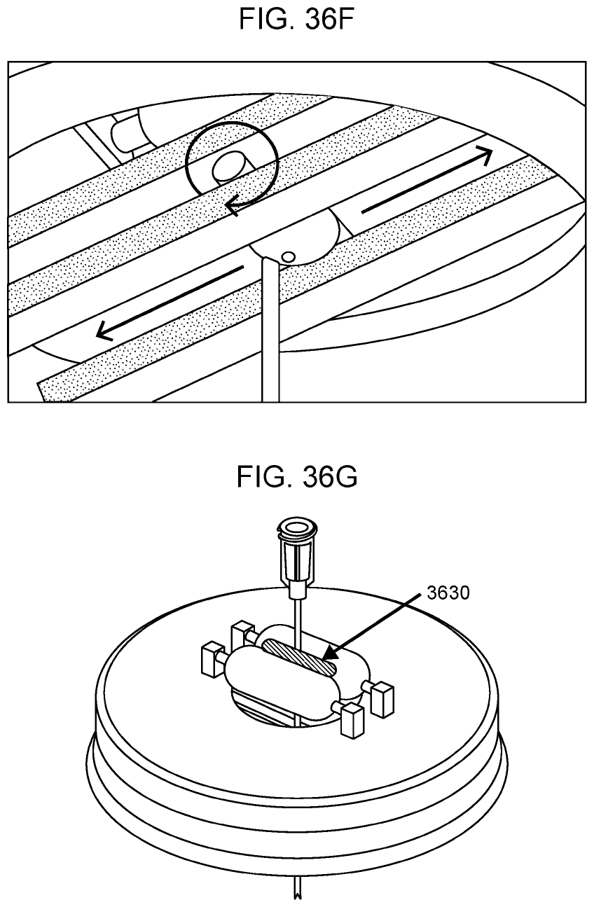

FIGS. 36A, 36B, and 36C show the assembled device and a needle inserted therethrough. In particular, upper housing 802 is placed at least partially within the base hole of base 801. Due to this placement, the principle axis passes through both the base hole and upper hole. The upper housing is configured to rotate around the principle axis in a manner that allows it to slide or rotate relative to the base. For instance, the base could be affixed to the skin of the patient whereas the upper housing can rotate. In the FIG. 37 embodiment, this rotation is achieved by placing ball bearings 808 between the upper housing and middle housing 807, wherein the middle housing is placed into direct contact with the inner circumference of the base hole. Thus, the base and middle housing remain stationary but the upper housing can rotate.

The device also includes a track comprising a first rail 803a and a second rail 803b. As shown in FIG. 38, each rail can be composed of two separate elongated members, as shown in the enlarged view of FIG. 39. Each rail has a first end and a second end that are each attached to the upper housing. The rails are also parallel to each other. Thus, rotation of the upper housing also causes rotation of the rails as a single unit.

The device further includes needle receiver 804 that includes a needle hole. The needle receiver of FIG. 38 is shown in enlarged format in FIG. 39. In this embodiment, the needle receiver has a central spherical section 804a with a needle hole 804b passing through its center. The needle receiver also has two cylindrical elongated members 804c and 804d (i.e. “arms”) extending outwards from the center. The circular cross section of the “arms” allow the needle receiver to rotate within the rails of the track. For example, FIG. 36C shows a bottom perspective of the assembled device. FIG. 36D shows an enlarged view of a section of FIG. 36C. Additionally, FIG. 36E shows an annotated version of FIG. 36D. Show is central spherical section 804a of the needle receiver. Also shown is arm 804c of the needle receiver that is positioned between the two elongated members of first rail 803a. Also shown is arm 804d positioned between the two elongated members of second rail 803b. Also shown is needle 820 passing through the needle hole and downwards. As shown in FIG. 36F the cylindrical shape of the arms allow the needle receiver to rotate within the rails. Also, the needle receiver can move along the rails because the arms can slide across the rails.

As shown in FIG. 36C-F, the needle can be inserted through the hole in the needle receiver. Due to the movement and rotation of the needle receiver, then precise angle of the needle can be advantageously controlled.

The movement of the needle receiver in the up, down, forward, and backward directions is limited by the rails of the needle receiver. In some cases, the movement is limited to 2 mm or less, such as 1 mm or less, 0.5 mm or less, or 0.1 mm or less. In contrast, the needle receiver can substantially move in the left or right direction along the rails. For example, the total left to right movement possible for the needle receiver can range from 1 cm to 15 cm. The needle receiver can rotate within the rails, e.g. the maximum rotation in one direction to the other direction can be between 15° and 150°.

The device further includes a needle aligner including first and second rollers, e.g. as shown as elements 805a and 805b in FIG. 37. Each roller includes a cylindrical section that is configured to rotate around a roller axis, wherein the roller axes are parallel to both each other and to the first horizontal axis. Thus, the rollers remain parallel to the first and second rails even when the upper housing is rotated relative to the base.

As used herein, the term “parallel” is used interchangeably with the term “substantially parallel”, e.g. wherein the items are within 5° of perfectly parallel, such as within 3° or 1° of perfectly parallel. It is understood that manufacturing procedures sometimes produce imperfect devices.

The rollers can be either connected directly to the upper housing or indirectly to the upper housing. As shown in FIGS. 36A and 36B, the rollers are attached to the cover 806, wherein the cover is attached to upper housing 802.

When assembled, the device also creates a space called the “roller-roller space” 830. This space is a horizontal plane of open space that is located: (i) between the first and second cylindrical sections of the rollers, (ii) directly above the space between the first rail and the second rail, (iii) directly above the upper hole, and (iv) directly above the base hole. For example, FIG. 36G is an annotated version of FIG. 36A that shows an approximation of roller-roller space 830. Roller-roller space 830 can be approximately referred to as the space between the rollers where the distal end of the needle is inserted between the rollers. Further movement of the needle downwards causes the distal end to enter the needle hole of the needle receiver. The angle and position of the needle receiver can match the angle and position where the needle passes through the roller-roller space. Together, these angles and positions fix the orientation of the needle. The rollers can also exert a compressive force on the needle that maintains the orientation of the needle and optionally inhibits needle movement downwards. Therefore, the needle only moves downwards in response to purposeful force from a surgeon.

Provided are methods of using the device. Provided is a method of surgically inserting a needle along a fixed three-dimensional orientation into a surgical region of a patient, the method comprising:
providing a surgical device for insertion of a needle along a fixed three-dimensional orientation;
fixing the base of the surgical device to a skin surface of the patient;
providing the needle, wherein a diameter at a middle section of the needle is less than a distance between the first roller and the second roller at the roller-roller space;
moving the needle receiver in a left or right direction along the track to a particular position;
rotating the needle receiver around the second horizontal axis to a particular orientation;
moving a distal end of the needle into the roller-roller space and then down past the roller-roller space, thereby causing the middle section of the needle to make and maintain contact with both the first roller and the second roller, wherein further motion of the needle causes the first and second rollers to rotate;
moving the distal end of the needle into the needle hole,
moving the distal end of the needle downwards and out of the needle hole; and
moving the distal end of the needle into the surgical region of the patient.

Also provided are kits containing the device and packaging containing the device. Optionally the kits further include the device and the needle. Provided are systems including the device and the needle.

### Systems

Also provided are systems that include the surgical device as described above along with one or more additional devices. For instance, the system can include the surgical device and one or more of: the controller, the display, the needle, and a spacer.

### Controller

For example, provided are systems that include a surgical device and the controller. In some cases, the controller is located within the housing of the surgical device. In other cases, the controller is located outside the housing, e.g. wherein the inertial measurement data is sent outside the housing and to the controller through a wired or wireless connection. As discussed above, the controller is configured to determine the relationships between the device's current pitch and yaw and the pitch and yaw of the desired trajectory by comparing an initial yaw, an initial pitch, the IMD, and the desired three-dimensional trajectory. The controller can also transmit the relationships between the device's current pitch and yaw to the desired pitch and yaw to the display, e.g. so that the display can visually indicate such information to the user.

### Display

Provided are systems that include a surgical device and the display. The display is configured to receive information from the controller regarding the relationships between the desired and current pitches and yaws. The display also visually indicates those relationships. For example, the display can show the different (e.g. in degrees) between the desired and actual yaw, and between the desired and actual pitch. For instance, the display can include a 7-segment unit to show each digit of the numerical differences. In some cases the display has a label indicating which number corresponds to the first dimension (e.g. pitch) and a second label indicating which dimension corresponds to the second dimension (e.g. yaw). For instance, such labels can be “pitch” and “yaw” or “x” and “y”. In some cases, the display indicates qualitative differences between the current and desired pitches and yaws.

In some cases, the display includes two or more lights of different colors that correspond to the relationship between actual and desired trajectories in the first dimension (e.g. pitch). During use, the display can illuminate a single light that corresponds to the current relationship between the actual and desired trajectories. For example, the display could comprise a red light and a green light. If the difference between actual and desired trajectories in the first dimension is acceptably small, then the green light can be illuminated, indicating an acceptable state to the user. However, if the difference is unacceptably large, then the red light can be illuminated, thereby informing the user. In some cases, the display can include a third light, e.g. and orange light, to indicate a mildly unacceptable angle, whereas the red light can be used to indicate a very unacceptable angle. In some cases, the lights can also indicate which direction the needle should be moved, e.g. there can be a left red light indicating the needle is too far left, a green light indicating an acceptable angle, and a right red light indicating the needle is too far right. Similarly, the display could include a red, orange, green, orange, and red light in sequence. Additionally, the angular relationship for the second dimension (e.g. yaw) can be indicated by a second set of two or more lights of different colors.

In some cases, the display is a “bull's eye” display. This type of display is a digital visual display, i.e. wherein there are many different pixels that form the overall digital display and can show a “picture”. This “digital visual display” is distinct from simply having a few different colored lights that indicate a “good” alignment with a green light or a “bad” alignment with a red light.

The bull's eye display comprises a digital visual display having at least 100 pixels, e.g. located on a top surface of the device. In some cases, there are at least 200 pixels, at least 400 pixels, or at least 800 pixels. In some cases, the digital visual display is a multicolor digital visual display, i.e. the pixels can emit two or more distinct colors, such as three or more, four or more, ten or more, or one hundred or more. This allows the digital visual display to provide information based on color and not simply based on the location of the illuminated pixels.

In some cases, the bull's eye display is configured to:
show a target orientation marker at the center of the digital visual display,
show a current orientation marker on the display based on the relationship between the current pitch and yaw and the desired pitch and yaw.

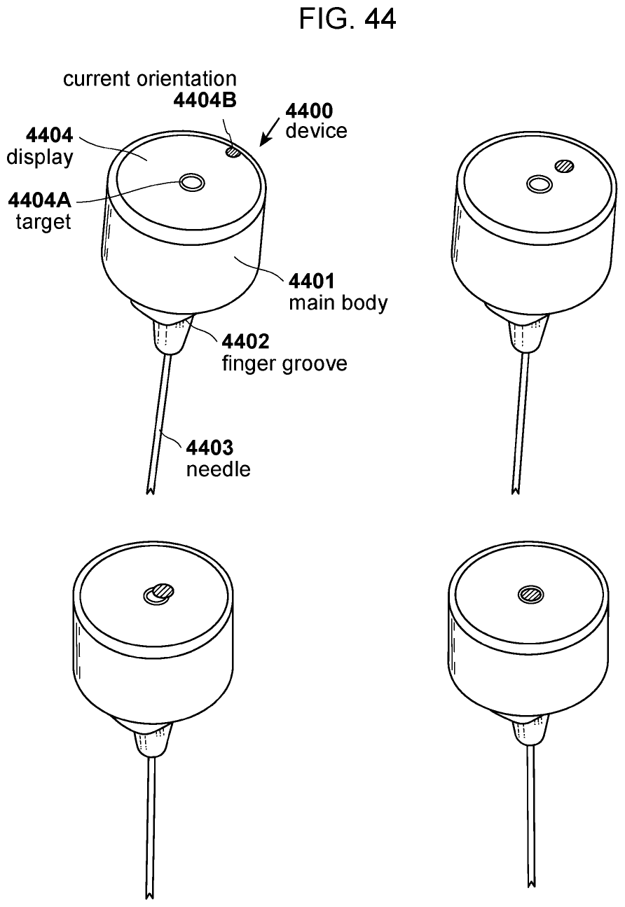

For example, FIG. 44 shows a bull's eye display wherein there is a central, hollow circle that represents the target orientation marker. Also shown are solid circles that appear in different locations of the display, which correspond to the current orientation marker. The first embodiment of this figure shows the solid circle adjacent to the edge of the display. This indicates that the current orientation is massively misaligned with the desired trajectory. This massive misalignment is also indicated by the red color of the solid circle.

Furthermore, the angular position of the solid circle (i.e. within a 360° circle) indicates which direction the device needs to be rotated. For example, carpenters are familiar with a “spirit level” (i.e. also called a “bubble level”) which is used to determine if a piece of wood is perfectly horizontal. The spirit level has a horizontal tube filled with liquid and one trapped air bubble. Since the air bubble is less dense than the liquid, horizontally tilting the spirit level will cause the air bubble to move to the higher end of the cylinder. The carpenter knows that the wood is perfectly level when the bubble is in the middle of the cylinder and located between two lines drawn on the cylinder.

Similarly, carpenters also use a “bull's eye spirit level” which determines if the piece of wood is perfectly horizontal in both horizontal directions. The bull's eye spirit level has a single air bubble trapped in a circular enclosure with liquid, and perfect horizontalness in both directions is achieved when the air bubble is located within a circle drawn on the top of the bull's eye spirit level. Since the air bubble rises to the higher side, the air bubble shows which side is too high.

The current “bull's eye display” can use the same principle as the “bull's eye spirit level” to indicate which side of the device is too high. Thus, the surgeon can rotate the device so that the solid circle moves into the center of the hollow circle, as shown in FIG. 44. Being inside the hollow circle indicates that the current trajectory matches the desired trajectory. FIG. 44 also shows that the solid circle changes from red (top-left) to yellow (top-right), green (bottom-left), and then blue (bottom-right) as it moves closer to the circle, giving another indication to the surgeon of how closely aligned the trajectories are.

In some embodiments, the display is attached to an external surface of the housing.

In other cases, the display is not attached to the external surface of the housing and is located elsewhere. For instance, the display can be a part of an indication apparatus that was specifically constructed for the purpose of communicating the pitch and yaw information.

In some cases, the display can be worn by the user. For example, a wearable indication apparatus can comprise the display and a band, e.g. so that the indication apparatus can be fastened around the wrist of a user. Stated in another manner, the methods can use an indication apparatus that is similar in form and function to a smart watch. For example, U.S. Pat. No. 9,307,917 is assigned to Fitbit LLC and describes a wearable fitness monitoring device. The shape, size, visual display, haptics, and electronic communication ability of a U.S. '917 device could be repurposed from a wearable device for fitness tracking to a wearable device for receiving information about the relationships between the desired and actual pitches and yaws.

In some cases, the display is a part of an electronic device built and constructed for a different purpose. For example, the pitch and yaw information can be communicated to a smartphone, and the smartphone display can be the same display used to perform the methods.

In some cases, the display comprises qualitative indication lights that provide a visual indication that one or more relationships are outside or inside a predetermined acceptable range. For example, if the actual pitch is more than 3° away from the desired pitch, then a red light can illuminate, informing that the pitch is unacceptably deviated from the desired pitch. In some cases, a green light can illuminate to inform the user that the actual pitch is within an acceptable range. In cases, such lights can blink, e.g. to provide a more noticeable signal to the user.

In some cases, the display is an augmented reality (AR) display. In some cases, the display is a virtual reality (VR) display.

In some cases, the system includes a haptic indicator that provides haptic information about the relationship between the desired and actual pitches and yaws. For example, the haptic indicator can vibrate to indicate such relationships. For instance, the haptic indicator can vibrate if the pitch, yaw, or both are outside a predetermined acceptable range.

### Audio Indicator

In some cases, the system includes an audio indicator that provides audio information about the relationship between the desired and actual pitches and yaws. For example, an auditory sound can be emitted with the pitch, yaw, or both are outside a predetermined acceptable range, thereby warning the user. In some cases, the audio indicator can emit sounds of different frequencies to indicate different relationships between the desired and actual angles, e.g. low frequency beeps to indicate poor agreement between desired and actual angles, and high frequency beeps to indicate good agreement between desired and actual angles. For instance, humans can generally hear sounds from about 20 Hz to 20 kHz in frequency. The sounds can also indicate which direction the needle should be moved in order to align it with the desired trajectory.

### Needle

In some cases the system includes a surgical device and the needle. For example, in some cases the needle ranges from a 34 gauge needle to a 10 gauge needle, e.g. wherein the outer diameter of the needle ranges from 0.159 mm to 3.404 mm. In some cases, the needle has a length ranging from 3 cm to 30 cm, wherein the length of the needle is the distance from the distal end of the needle to a proximal end. In some cases the distal end of the needle has a slanted opening, which can make it easier to pierce skin and body tissues.

### Additional Aspects

In some cases, the system includes the surgical device, the display, and the needle. In some cases, the system further includes the controller. In some cases, the system further includes a spacer.

Examples of additional devices that can included in the system are: a light sensor, a color sensor, a distance sensor, a spectroscopic sensor, an EKG sensor, a flow sensor, a pulse oximeter, an ultrasound sensor, a doppler sensor, a glucose monitor, a respiration sensor, and a point of care diagnostics sensor.

In some cases, the system includes a distance sensor. For instance, the distance sensor can be attached to a bottom surface of the housing of the surgical device. As such, the distance sensor can measure the distance to the skin of the patient, thereby providing additional information that can help the user know the position of the needle relative to the patient. In some cases, the distance sensor is a laser range finder. For instance, Forrester and Hulme provide a review of the principles of laser range finders and some of the applications (Optical and Quantum Electronics, 1981, 13, 259).

In some embodiments, the system is configured to electronically communicate with an electronic medical record (EMR). In some cases, this communication can be performed through wireless communication protocols. For example, the method could include sending and receiving data from a medical imaging archive device.

### Methods

Provided by the present disclosure are methods of using a surgical device to guide insertion of a needle along a desired three-dimensional trajectory from a surgical entry site to a surgical destination site. In some cases, the method includes:
selecting the desired three-dimensional trajectory;
providing a system comprising:
   a surgical device as described herein;
   a controller;
   a display that receives and visually indicates the relationships between the current pitch and yaw of the device and the pitch and yaw of the desired trajectory; and
   a needle;
orienting the device such that the spatial orientation indicator is aligned with an initial reference;
notifying the controller that the spatial orientation indicator is aligned with an initial reference, thereby causing the controller to determine that the device has an initial pitch and an initial yaw, and thereby also causing the controller to monitor the inertial measurement data (IMD) and transmit the relationships between the device's current pitch and yaw to the desired pitch and yaw to the display;
moving the device to the surgical entry site;
observing the display and adjusting the pitch and yaw of the device such that the actual trajectory is more closely aligned with the desired trajectory;
if a section of the needle is not already positioned within the hole, moving the needle such that a section of the needle is positioned within the hole; and
moving the needle such that a distal end of the needle is inserted into the surgical entry site along the actual trajectory to a first depth.

As discussed above, the desired trajectory begins at the surgical entry site and ends at the surgical destination site. For example, the entry site can be a particular location on the skin and the destination can be a location for therapeutic agent delivery or for the collection of a biological sample. For example, a liquid solution comprising an active pharmaceutical ingredient (API) can be delivered through the needle to the destination site. For example, a biological sample of a suspected cancerous tumor can be collected through the needle from the destination site.

In some cases, the desired trajectory consists of a single line segment from the entry site to the destination. As such, the desired trajectory consists of a single desired pitch and a single desired yaw.

In other cases, the desired trajectory includes two or more line segments, e.g. in order to avoid in impassable region such as bone, to avoid an undesirable location such as a major blood vessel, or both. As such, the desired trajectory will include two or more desired pitches and two or more desired yaws. The method involves moving the distal tip of the needle towards a first intermediate location along a first pitch and a first yaw, and then moving the distal tip towards a subsequent location along a second pitch and a second yaw. In some cases, the subsequent location is the destination site.

In some cases, selecting the desired three-dimensional trajectory includes:
recording a plurality of two-dimensional (2D) images of the surgical region with a medical imaging device;
generating a three-dimensional (3D) representation of the surgical region from the plurality of 2D images;
using the 3D representation to choose the desired trajectory from a surgical entry site to a surgical destination site.

In some cases, using the 3D representation to choose the desired trajectory desired trajectory is performed by a human user. In some cases, a human user instructs a computer algorithm to choose the desired trajectory based on the 3D representation, i.e. choosing the desired trajectory is performed by a software algorithm. In some embodiments, choosing the desired trajectory comprises avoiding an undesirable region (e.g. a major blood vessel) or avoiding an impassable region (e.g. bone). Avoiding an undesirable region can also be referred to as avoiding anatomic structures. Such regions include, for example, bone, blood vessels, airways, the heart, and bile ducts. In some embodiments, the number of 2D images used to generate the 3D representation ranges from 2 to 264, such as from 10 to 100.

The method includes providing a system comprising a surgical device as discussed above, a controller, the display, and the needle.

The method also includes orienting the device such that the spatial orientation indicator is aligned with an initial reference. For example, the initial reference can be a grid of lights from a medical imaging device, e.g. a CT scanner, a PET scanner, or an MRI scanner. In such cases, the axes of the medical imaging device can be used as the reference axes, e.g. the x axis, y axis, and z axis.

The method includes notifying the controller that the spatial orientation indicator is aligned with an initial reference. For example, such notification can comprise activating a calibration button on an exterior of the surgical device that is configured to send an electronic notification to the controller that the spatial orientation indicator is aligned with the initial reference. In some cases, the notification is sent directly from the calibration button to the controller. In some cases, the notification is sent from the calibration button to the electronic communication element, which then sends the notification to the controller. In cases wherein the notification button is used, the surgical device comprises the notification button on the exterior of the surgical device. In other cases, the notification comprises using a graphical user interface, e.g. on a tablet computer or a laptop computer, to send such notification to the controller.

Such notification of the controller causes the controller to determine that the device has an initial pitch and an initial yaw. Afterwards, the inertial measurement unit will send inertial measurement data to the controller, thereby allowing the controller to continually update the current pitch and yaw of the device. As such, the controller will also transmit the relationships between the desired pitch and yaw and the current pitch and yaw to the display. Hence, the display will visually display the current relationship between the current and desired pitches and yaws. For example, the display can visually show the numerical different in degrees between the current and desired pitches and yaws. The display can also show the difference as either positive or negative to indicate whether the angle is too far in one direction or whether the angle is too far in the opposite direction. In some cases, the display is attached to the surgical device, e.g. attached to a surface of the surgical device facing away from the subject. In some cases, the display is separate from the surgical device, i.e. it is not attached to the surgical device. In some cases the display is a digital visual display, e.g. the bull's eye display as described herein.

After calibration to the initial reference, the surgical device is moved to the surgical entry site. In some cases the device is moved a distance ranging from 1 cm to 20 m, such as 5 cm to 5 m.

The method further includes observing the display and adjusting the pitch and yaw of the device such that the actual trajectory is more closely aligned with the desired trajectory. Hence, if the display shows the numerical deviation from the desired trajectory, then the user adjust the orientation (i.e. pitch and yaw) of the device such that the numbers shown on the display become smaller. In some cases, the observing and adjusting can be performed simultaneously, i.e. wherein the user adjusts the orientation while looking at the display. In other cases, the observing and adjusting are performed sequentially, i.e. wherein the user observes the display, adjusts the orientation, and then observes the display a second time. In some cases, the observing and adjusting results in an actual pitch that is 5° or less from the desired pitch, such as 3° or less or 1° or less. In some cases, the observing and adjusting results in an actual yaw that is 5° or less from the desired yaw, such as 3° or less or 1° or less. In some cases, the observing is of a digital visual display, and the user changes the orientation of the device so that the current orientation marker meets the desired orientation marker.

Additionally, if a section of the needle is not already positioned within the hole upon completion of the observing and adjusting, then the needle is moved so that a section of the needle is positioned within the hole. Stated in another manner, in some cases a section of the needle is already positioned within the hole during the observing and adjusting. In other cases, all sections of the needle are outside the hole during the observing and adjusting, and therefore after the observing and adjusting the needle is moved such that a section of the needle is within the hole.

Afterwards, the needle is moved such that a distal end of the needle is inserted into the surgical entry site along the actual trajectory to a first depth. In some cases, the needle and surgical device are in a fixed spatial relationship, and therefore both the needle and surgical device are moved together. For example, a force can be exerted on the housing that causes both the surgical device and the needle to move in the same direction and distance. In other cases, the needle and surgical device can move relative to each other, e.g. wherein a force is exerted on the needle and the needle moves towards the surgical destination site without movement of the surgical device. In some cases, the display is observed while moving the needle, e.g. to verify that a proper trajectory is maintained. In other cases, the display is not observed while moving the needle. As well, in some cases, the distance from the device to the patient's skin surface is continuously measured, thus allowing for precise monitoring of the actual needle positioning in the patient.

In some cases, moving the needle to the first depth brings the needle to the surgical destination site. As such, the next step in the procedure can be performed, e.g. administering an active pharmaceutical ingredient or collecting a biological sample. In some cases, moving the needle to the first depth does not bring the needle to the surgical destination site.

In some cases, the method further includes recording an image of the inserted needle at the first depth. For example, the image can be a CT scan, a PET scan, or an MRI scan. As such, the current location of the distal end of the needle can be determined.

Afterwards, the needle can be moved along a second trajectory to a second depth. In some cases, the desired second trajectory has the same pitch and yaw as the initial trajectory, i.e. the needle simply needs to be moved further in the same direction. In such cases, the display can be observed and adjustments to the orientation can be made, if desired, before inserting the needle to the second depth.

In other cases, the desired second trajectory has a difference in the pitch, yaw, or both compared to the first desired trajectory. For example, the overall route could include two different line segments in order to avoid an undesirable region such as a major blood vessel. As such, the method can include selecting a first updated trajectory, adjusting the pitch and yaw according to the display in order to more closely align with the first updated trajectory, and then moving the needle such that the needle is inserted to a second depth into the surgical region along the first updated actual trajectory.

Such steps can be repeated any necessary number of times until the surgical destination site is reached by the distal end of the needle.

In some cases, there is a hole in the housing of the device and the needle attachment element is a needle mount attached to the housing. For example, the needle mount can include multiple screws that contact the needle and hole it in a desired position relative to the device.

In embodiments with the needle mount, the device is moved to the destination site and is then securely attached in a particular orientation, such as by gluing the device the patient's skin. Thus, the device has a known spatial orientation. Afterwards, the needle mount can be adjusted such that the needle is in the correct orientation.

For example, the line passing directly through the hole of the device can be at 45° relative to the floor of the surgical room. Therefore, if the needle passes directly through the hole, it will also be 45° relative to the floor. However, the desired trajectory might be 35°. Therefore, the screws of the needle mount can be adjusted so that the needle is oriented at-10° relative to the hole. Therefore, the needle will be correctly oriented at 35° relative to the floor.

As such, in embodiments with the needle mount, the method does not include the step of “observing the display and adjusting the pitch and yaw of the device such that the actual trajectory is more closely aligned with the desired trajectory”. Instead, the method includes the steps of “attaching the needle to the needle mount” and “observing the display and adjusting the needle mount such that the actual trajectory of the needle is more closely aligned with the desired trajectory”.

### Controller

Also provided by the present disclosure are controllers for performing the methods described herein. The term “controller” and “computer controller” are used interchangeably herein. The controller can include a various hardware modules such as a processor (e.g. a microprocessor) and a computer memory module (e.g. random access memory (RAM)).

In some cases, the controller is configured to:
determine a desired pitch and a desired yaw based on the desired three-dimensional trajectory; and
receive notice that the spatial orientation indicator is aligned with an initial reference and therefore the device has an initial pitch and an initial yaw;
receive the inertial measurement data (IMD) from the electronic communication element; determine the device's current pitch and current yaw based on the initial pitch, the initial yaw, and the inertial measurement data;
transmit the relationships between the device's current pitch and yaw to the desired pitch and yaw to a display.

The controller is configured to determine the desired pitch and yaw based on the desired 3D trajectory. In particular, the desired 3D trajectory includes a route from the surgical entry site to the surgical destination site. A certain coordinate system (e.g. x, y, and z axes) will be used, such as the coordinate system of a CT scanner or other medical imaging device that is located near the patient. For example, pointing along the x axis in the positive direction could be considered to have a pitch of 0°and a yaw of 0°. By starting from the reference coordinate system and knowing the position of the patient relative to the reference coordinate system, along with the route from the entry site to the destination site, the determine the desired pitches and yaws. If the route has a single line segment, then there will be a single pitch and yaw. If the route has multiple line segments, e.g. to avoid blood vessels, then there will be first pitches and yaws along with subsequent pitches and yaws.

As discussed above, in some cases the notification of spatial orientation indicator alignment is received from a calibration button on the exterior of the device that is pressed by a user. In some cases, the notification is received from a laptop computer, desktop computer, tablet computer, or other device where a user caused the notification to be sent through a graphical user interface. As such, the controller determines the initial pitch and the initial yaw of the device relative to a reference coordinate system, e.g. the coordinate system of a CT scanner or other medical imaging device.

Furthermore, the controller receives the inertial measurement data and determines the device's current pitch and yaw from the initial pitch, the inertial measurement data. The current pitch and yaw can also be referred to as the current orientation or the current three-dimensional orientation. The current pitch and yaw can be updated each time the controller receives inertial measurement data from the IMU. As such, in some cases the time interval between determinations of the current pitch and yaw by the controller range from 0.01 ms to 1 second, such as from 0.1 ms to 500 ms or from 1 ms to 100 ms. The reception of intertial measurement data and determination of current orientation can be repeated for any suitable interval of time, such as from 1 minute to 10 hours, such as from 5 minutes to 2 hours.

The controller also transmits the relationships between the device's current pitch and yaw to the desired pitch and yaw to a display. For example, the controller can determine the different between the current and desired pitches by subtracting the current pitch (e.g. in degrees) from the desired pitch. Similarly, the current and desired yaws can be subtracted from one another to give the deviation from the desired yaw. Thus, in some cases the controller transmits the numerical deviation between the desired and current pitches and yaws to the display.

In some embodiments, the display is a digital visual display, e.g. a bull's eye display, as described herein. As such, the controller instructs the display for which pixels to illuminate and optionally in which color. This illuminated pixels correspond to how badly the trajectory is misaligned and what is the angle of misalignment (i.e. from 0° to 360°).

Also provided is a non-transitory computer readable storage medium with computer executable instructions stored thereon executed by a processor to perform a method of using a surgical device to guide insertion of a needle along a desired three-dimensional trajectory from a surgical entry site to a surgical destination site. The method can be any of the methods described herein. The computer executable instructions can be executed on the controller.

### Kits

Also provided by the present disclosure are kits. In some cases, the kit includes a surgical device as described herein along with packaging containing the surgical device. In some cases the kit further includes one or more additional devices, e.g. the display, the controller, the needle, one or more spacers, or a combination thereof. In some cases, the kit comprises a In some cases, the packaging comprises extruded polystyrene foam (XPS), bubble wrap, or a combination thereof.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for.

### Example 1

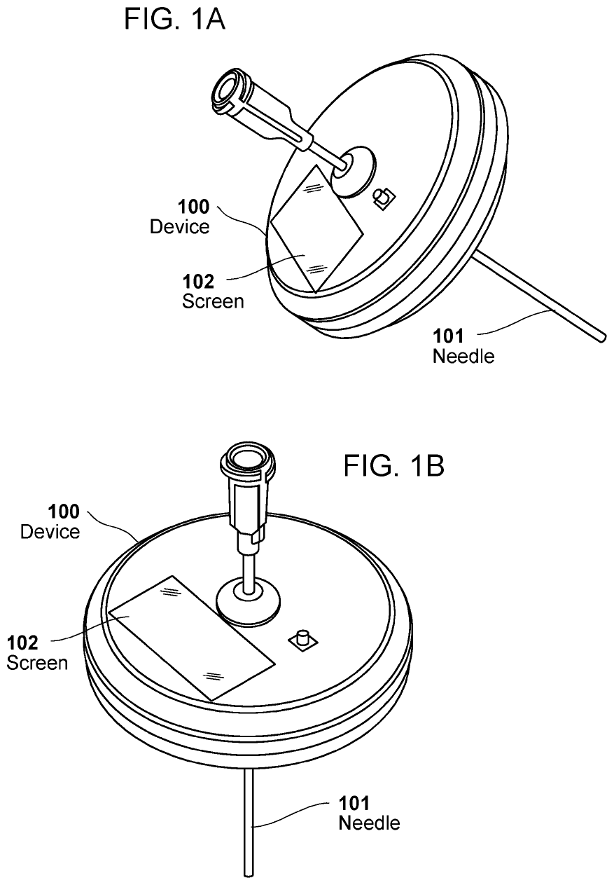

A surgical device for guided insertion of a needle was designed, as shown in FIGS. 1A and 1B. The housing had the overall shape of a short cylinder with rounded edges. Inside the housing was an insertion measurement unit (IMU) and an electronic communication element that could transmit data from the IMU to a controller for analysis and comparison with the desired trajectory. Additionally, the top side of the device included a small cylindrical protrusion and a rectangular region for the attachment of a visual display that can indicate the relationship between the current and desired pitches and yaws. The housing included a hole through its center and an insert positioned within the hole. As shown in FIGS. 1A and 1B, a needle was inserted through the hole and the insert.

### Example 2

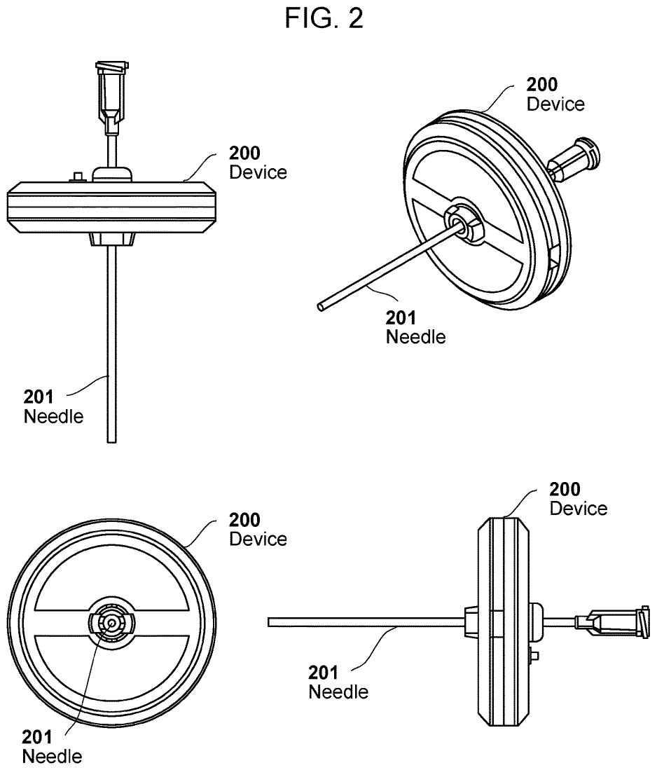

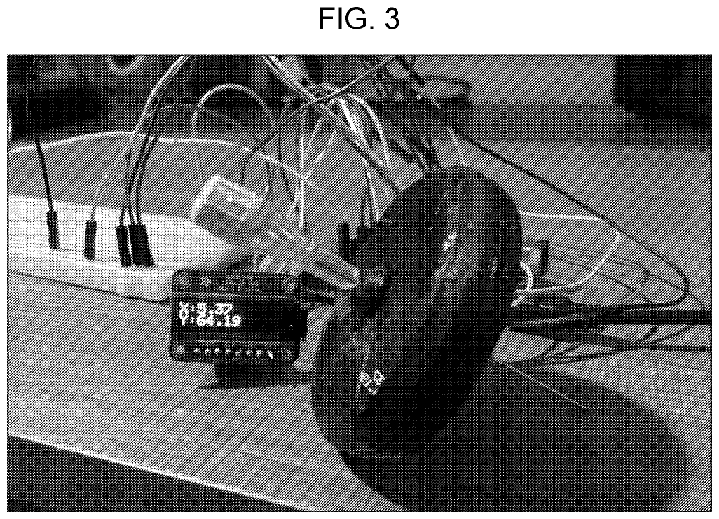

As shown in FIG. 2, a second surgical device was designed, wherein the height of the housing was 0.5 inches and the outer diameter of the housing was 2 inches. As shown in FIG. 3, a device of such shape and dimensions was constructed. FIG. 3 also shows several wires connecting the device to a controller and to a visual display. The visual display is located separately from the device shows an “x” value of 5.37 and a “y” value of 64.19, which represent pitches and yaws in degrees.

### Example 3

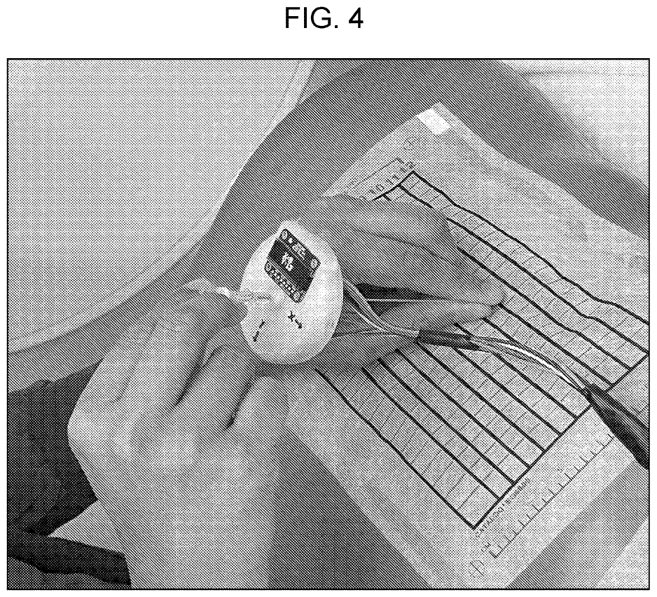

FIG. 4 shows a device with a visual display that is mounted to the housing of the device.

FIG. 4 shows the hands of a surgeon that has placed the end of the needle at a particular location on a surgical grid corresponding to the surgical entry point. The top side of the housing also has markings that show the direction of the “x” and “y” axes. As such, the surgeon was able to keep the distal tip of the needle at the surgical entry site while moving the top of the needle, thereby adjusting the pitch and yaw of the device until it matched the pitch and yaw of the desired trajectory.

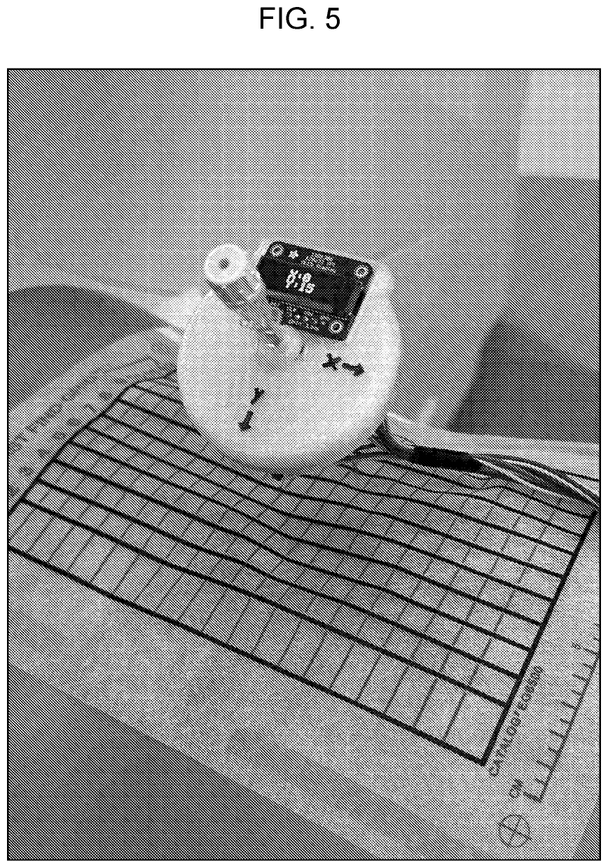

FIG. 5 shows the same device and needle as FIG. 4 after the device has been moved in order to achieve an “x” angle of 0 degrees and a “y” angle of 15 degrees.

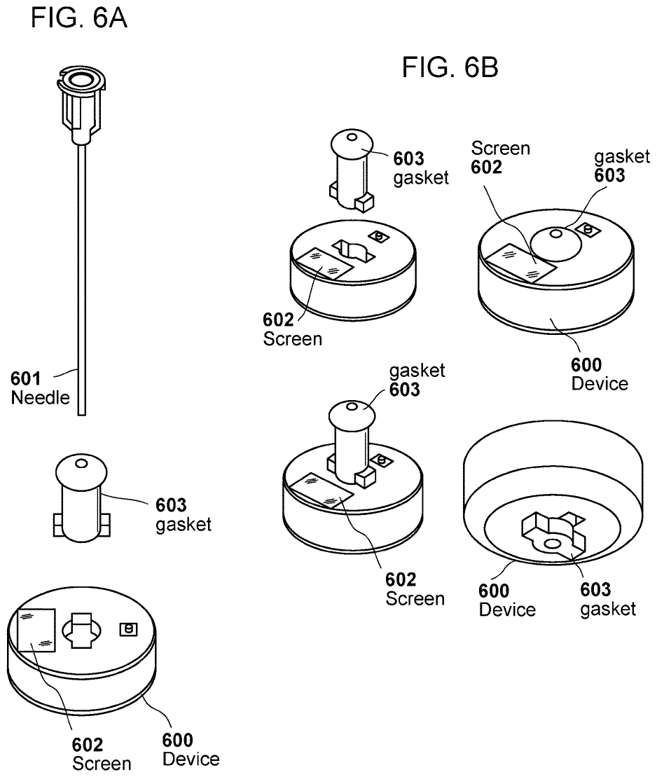

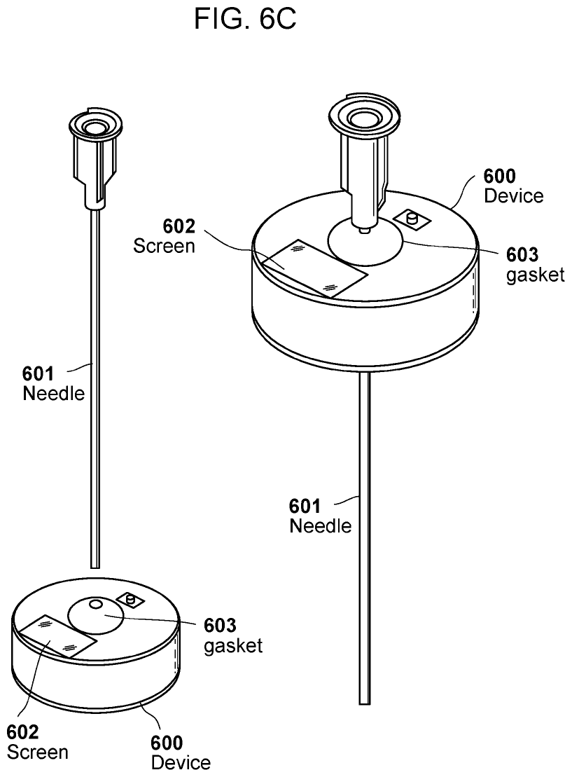

FIG. 6A shows an exploded view of a housing, an insert, and a needle. FIG. 6B shows the insert being placed into the hole and rotated in order to lock it into position. FIG. 6C shows the needle being inserted into the insert, which is located in the hole.

### Example 4

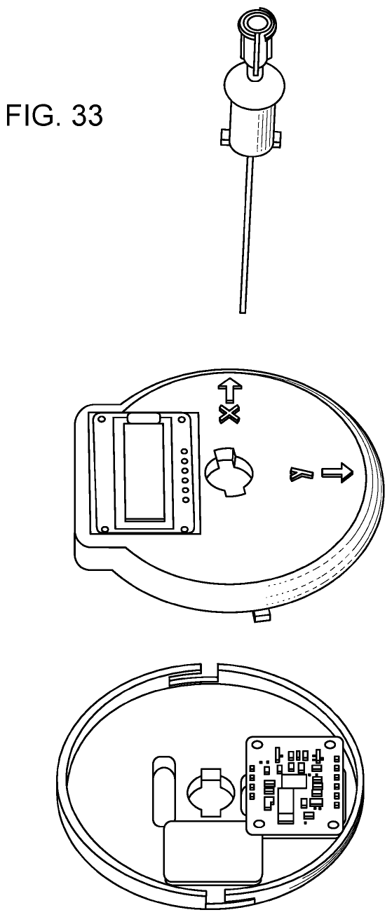

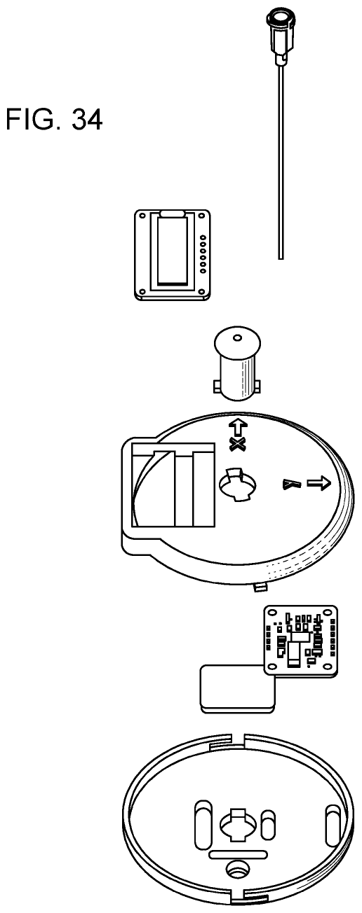

FIG. 33 shows an exploded view of a surgical system. The system includes a bottom section of the housing with electronic components placed thereon. Also shown s a top section of the housing with an “x” and “y” indicators of direction. Also shown is a display that gives can give the angel (e.g. in degrees) corresponding to the x and y directions. Also shown is a needle positioned with a spacer which has two flanges, e.g. so that it can be rotated and locked into position within the hole of the device.

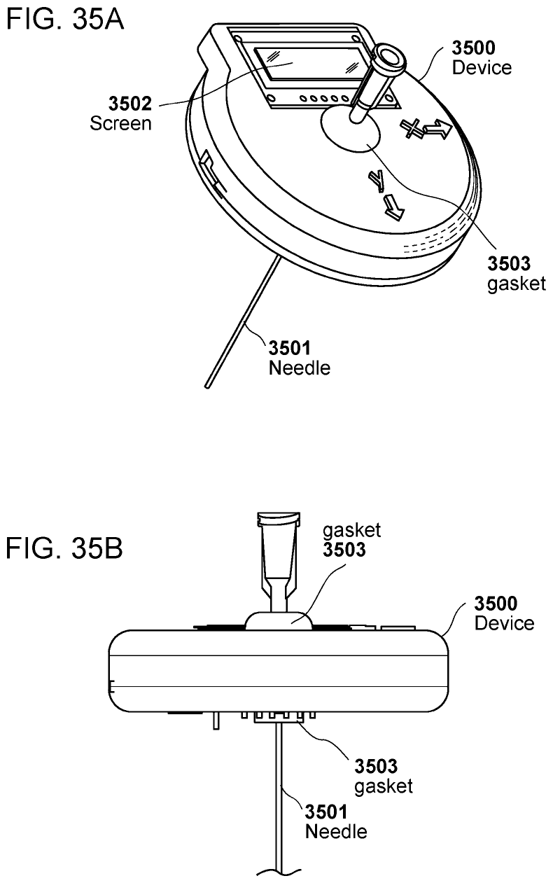

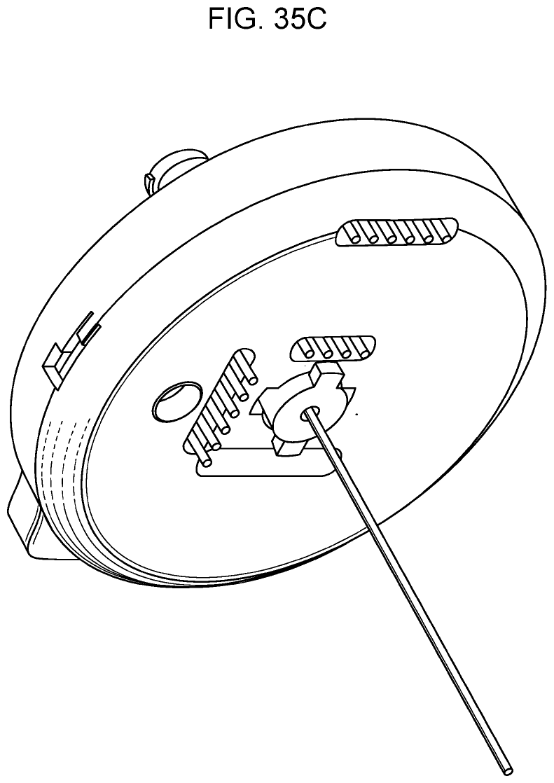

FIG. 34 shows an exploded view of FIG. 33 wherein each individual component is shown separately. FIG. 35A shows a perspective view of the assembled system of FIGS. 33 and 34. FIG. 35B shows a side view of the FIG. 35A system.

### Example 5

Additional devices were designed that include additional elements for performing other useful functions related to the surgical insertion of the needle.

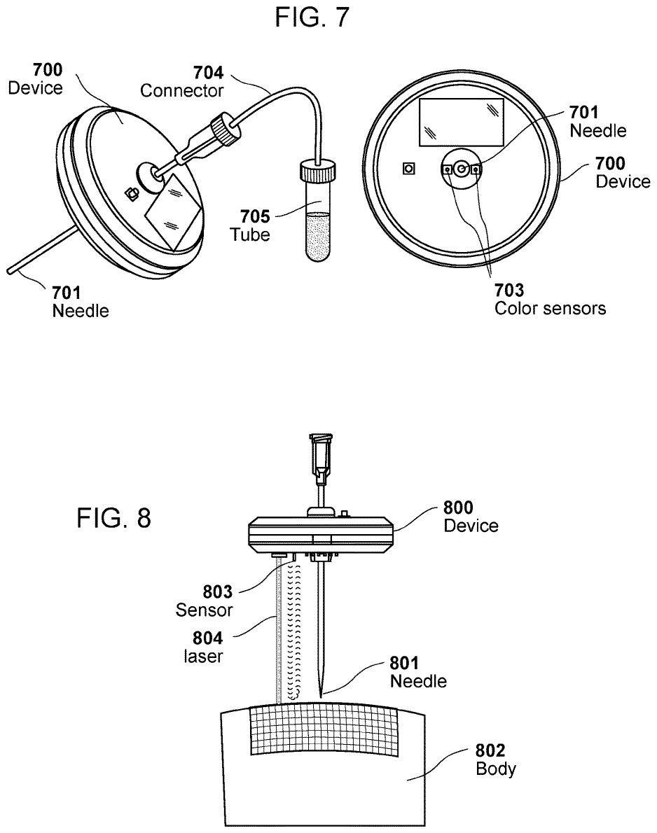

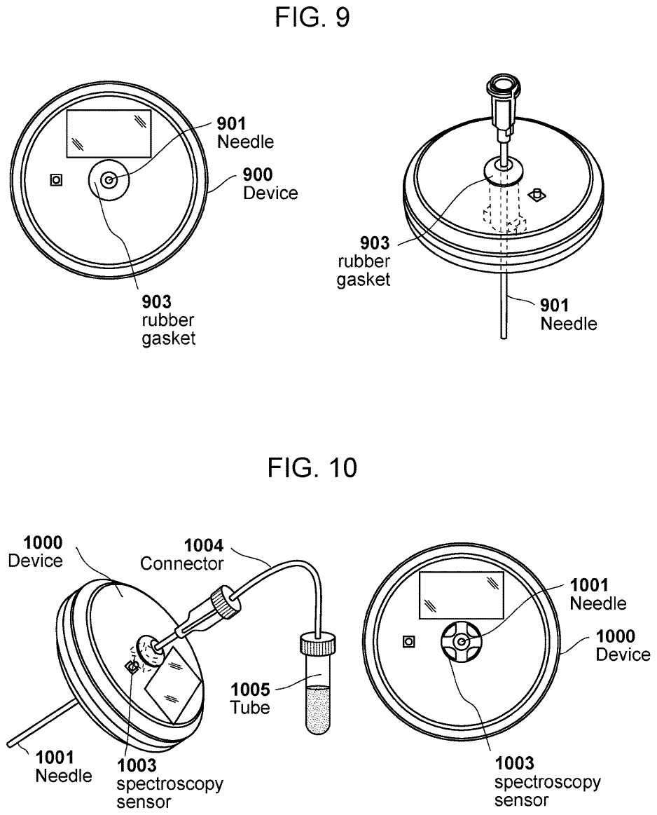

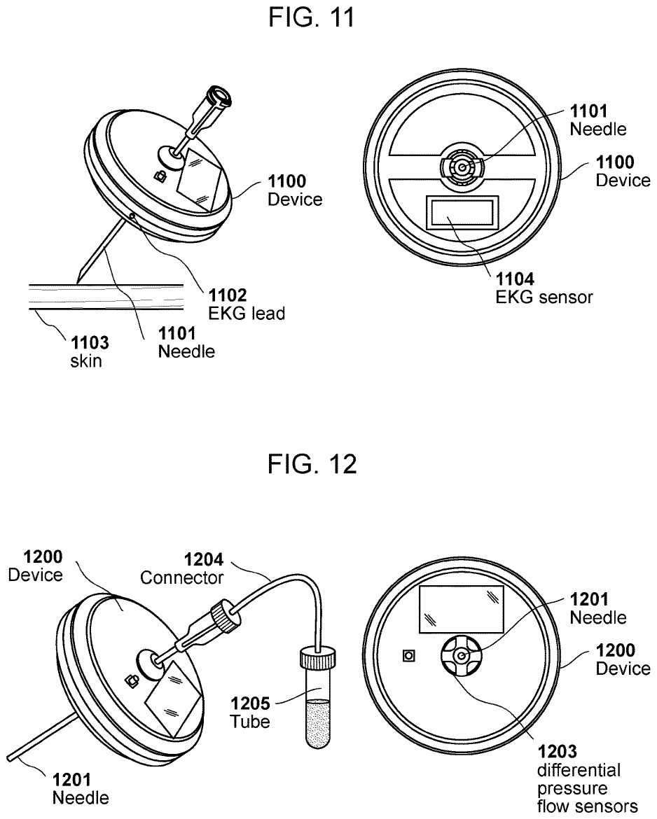

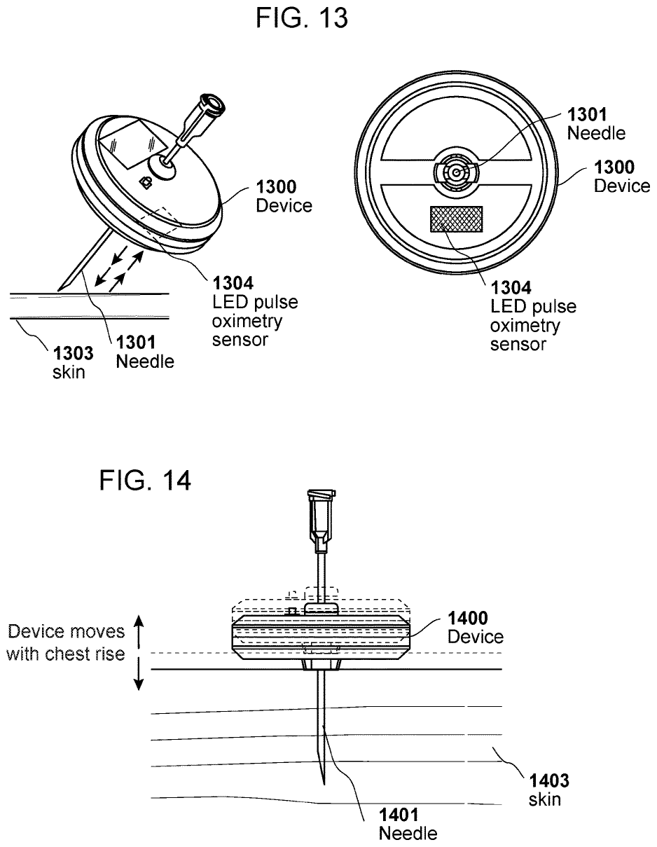

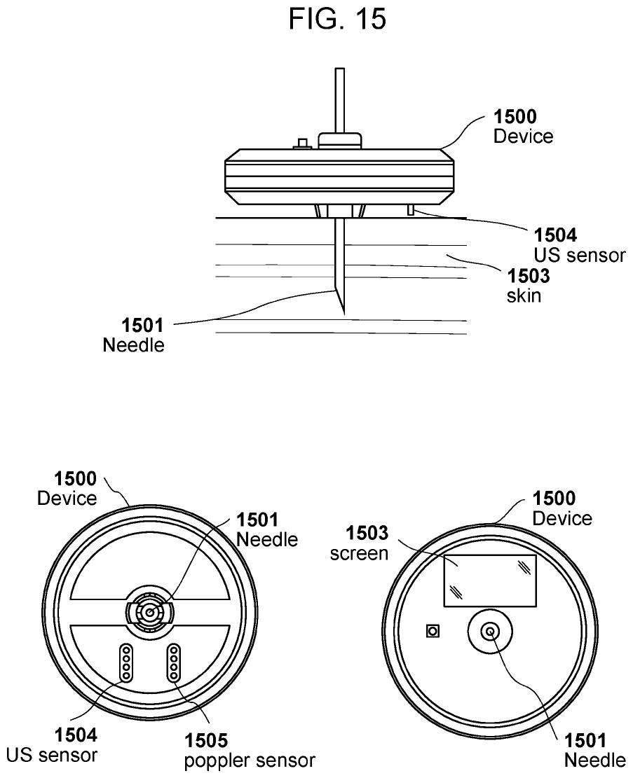

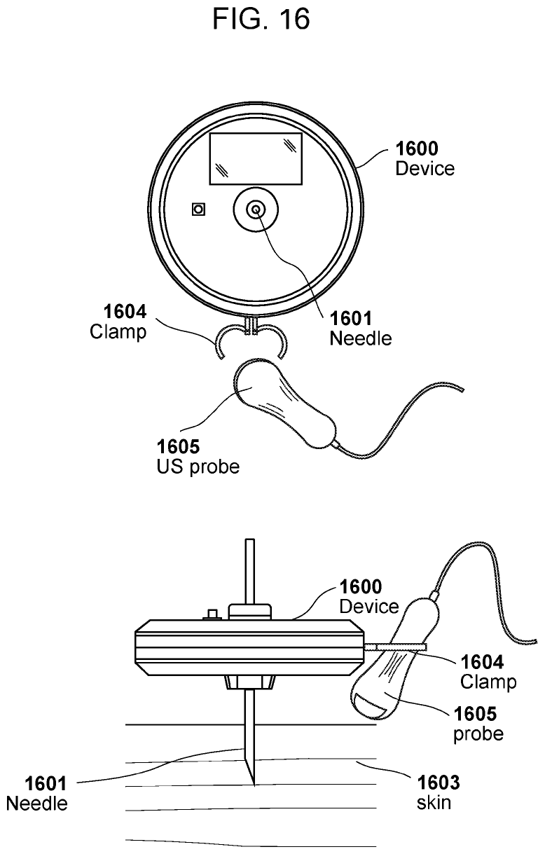

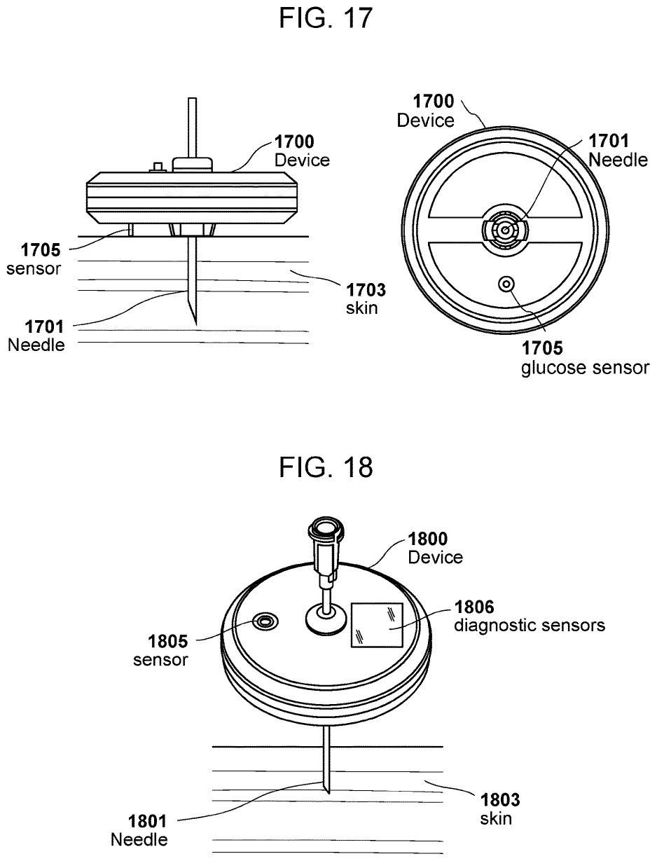

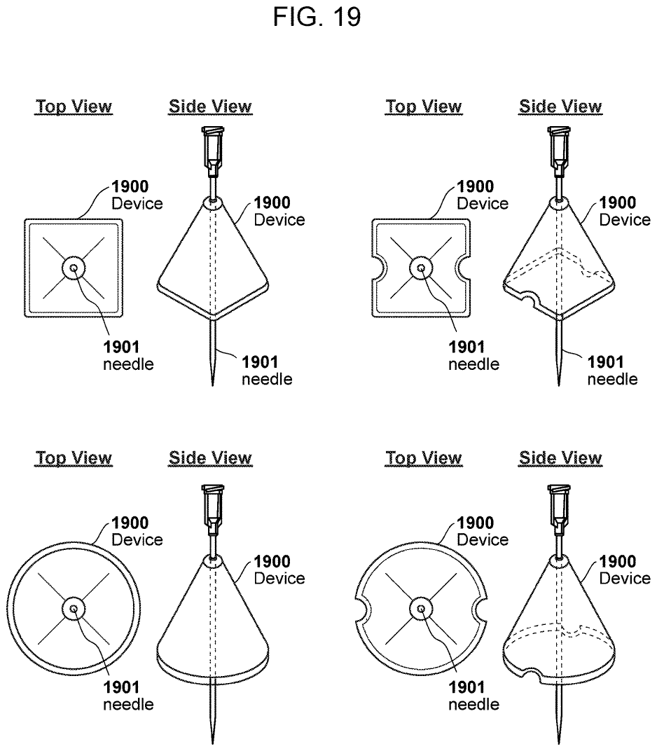

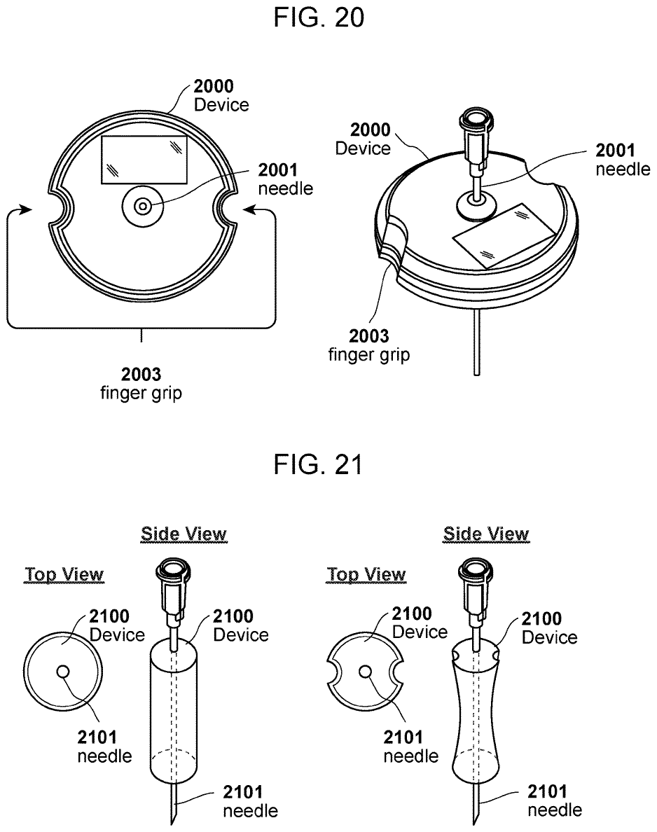

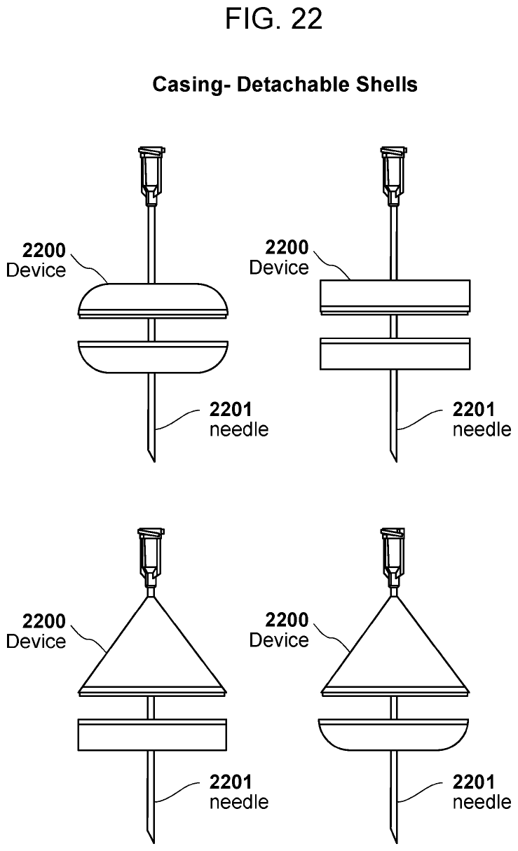

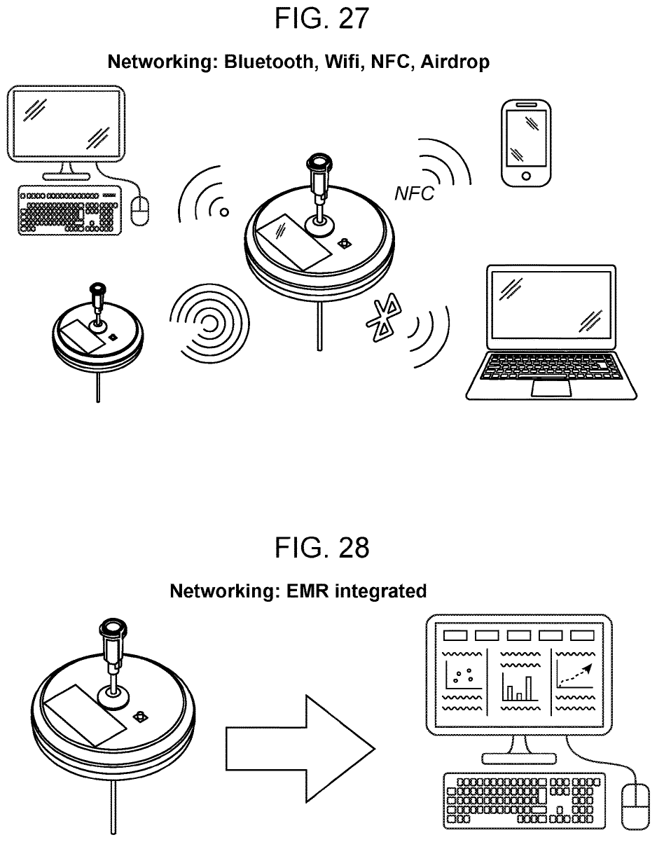

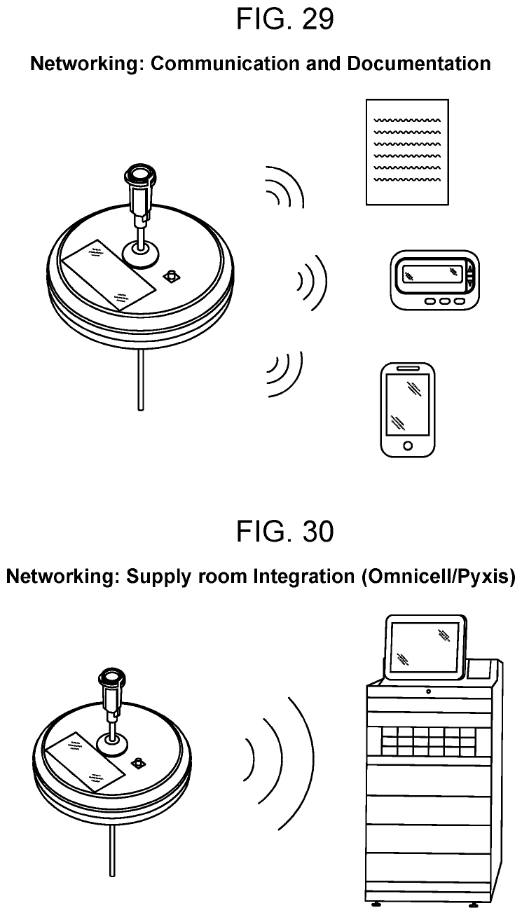

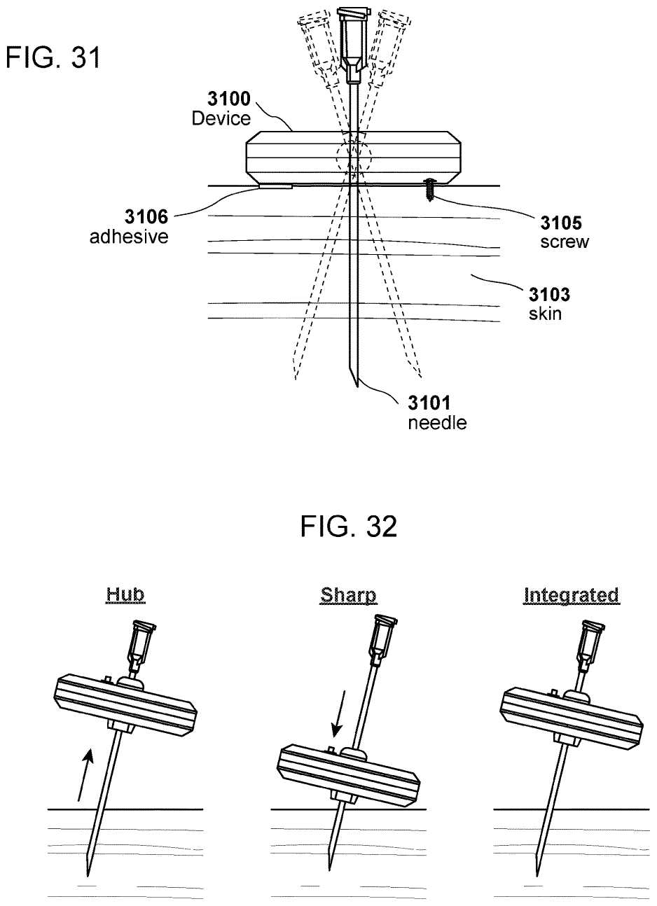

FIG. 7 shows a light and color sensor that is able to detect the color of the fluid as it passes through the center of the device. FIG. 8 shows a device with a sensor on the bottom of its housing that emits a laser that can be used to detect the distance between the bottom of the device and the top surface of the body of the patient. FIG. 9 shows different options for securing the needle in the hole, e.g. there can be a rubber gasket, a self-tightening twist lock, a magnetic, or an adhesive on the inner circumference to hole the needle in place. FIG. 10 shows a spectroscopic sensor that is able to detect the chemical composition of a fluid as it goes through the needle and passes through the center of the device. FIG. 11 shows a device with an EKG that assesses the patient. FIG. 12 shows a flow sensor and the device. FIG. 13 shows a pulse oximeter and a device. FIG. 14 shows a respiration sensor. FIG. 15 shows an ultrasound or doppler sensor. FIG. 16 shows a second ultrasound or doppler sensor. FIG. 17 shows a glucose monitor. FIG. 18 shows a point of care diagnostics sensor. FIG. 19 shows housings that have shapes other than a circular “donut” shape. FIG. 20 shows an approximately cylindrical housing with “finger grip” sections that are indentations that can be used to receive the fingers of a surgeon, thereby increasing the dexterity of the device. FIG. 21 shows additional shapes that can be used to increase dexterity and increase the ability of the surgeon's hand to easily control the orientation of the device. FIG. 22 shows detachable shells, wherein the device can have the ability to mix and match the bottom and top parts of the shells. FIG. 27 shows the ability of the device and system to connect via BlueTooth, WiFi, NPC, and Android communications. FIG. 28 shows the connection to an electronic medical record (EMR). FIG. 29 shows the networking ability of the devices and systems, allowing them to communicate information and notifications to other devices, such as smartphones and pagers. FIG. 30 shows that the devices and systems can integrate and communicate with a supply room software. FIG. 31 shows an embodiment wherein the housing is attached to the skin of the patient with adhesive. As such, insertion of the needle to the surgical destination site is performed by moving the needle relative to the housing. FIG. 32 shows an embodiment wherein the needle is in a fixed relationship with the housing, and the housing is not fixed to the skin. Thus, the needle is inserted by moving both the needle and the housing as a single unit, e.g. wherein a fastener retains the relative positions of both objects.

### Example 6: Guidance Along a Fixed Three-Dimensional Orientation

FIG. 36A shows a top perspective view of a surgical device for guiding insertion of a needle along a fixed three-dimensional orientation, along with a needle. FIG. 36B shows a side view of the FIG. 36A system. FIG. 36C shows a bottom perspective view of the FIG. 36A system. FIG. 37 shows an exploded view of a surgical device for guiding insertion of a needle along a fixed three-dimensional orientation. FIG. 38 shows an exploded the FIG. 37 device wherein all of the components are fully separated.

### Example 7: Bottom Needle Attachment Element

FIGS. 40A-40E show different views of a device with a bottom needle attachment element (BNAE). FIG. 40A shows a top view with calibration buttons 4005 along with display 4004, wherein the diameter is 44 mm. FIG. 40B shows a side view with main body 4001 (i.e. a first side surface) and finger groove 4002 is a finger receiving location that comprises a second side surface. In FIG. 40B the second side surface is curved and concave out. This allows the user to position their fingers in the groove of the finger receiving location, thereby aiding in the secure and easy handling of the device, e.g. as shown in FIGS. 41B-C. FIG. 40C shows a perspective view of the device while attached to a needle along with a United States Quarter Dollar Coin, which has a diameter of 0.955 inches (24.26 mm) and a thickness of 1.75 mm according to the U.S. Mint. FIGS. 40D-E shows an internal cross-section showing various electronic components.

FIG. 41A shows the device with main body 4101, finger groove 4102, and needle 4103.

FIG. 41B shows a user positioning the end of the needle along a grid that simulates the body of a patient. FIG. 41C shows an enlarged view of the device of FIG. 41B.

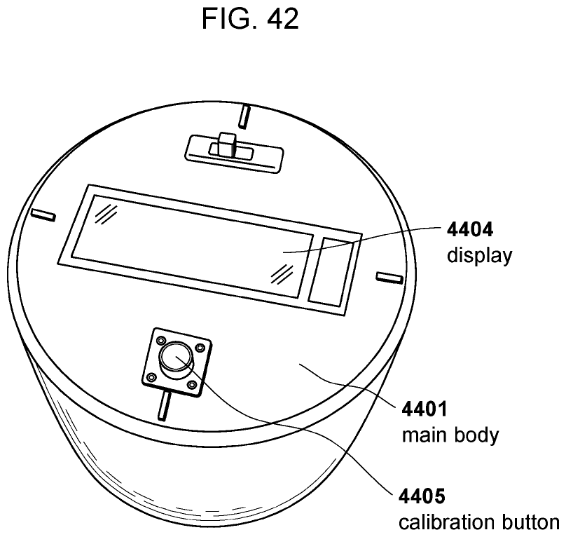

FIG. 42 shows a device with main body 4401, display 4404, and calibration button 4405.

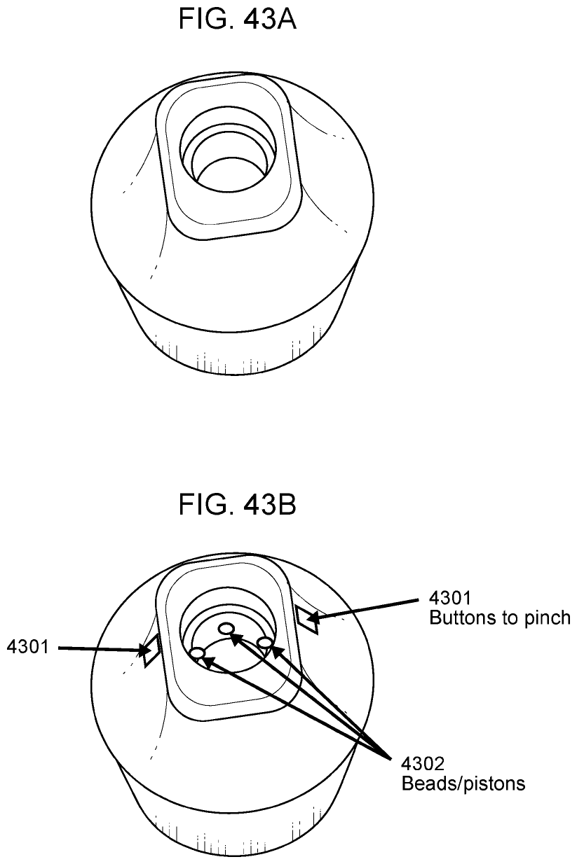

FIGS. 43A-B show a bottom perspective view of a pinching type BNAE. The BNAE includes buttons 4301 on opposite sides of the BNAE for pinching the needle. The device also includes plungers 4302, which can be referred to as “beads” if spherical or “pistons” if cylindrical.

FIG. 44 shows four view of a device with a circular digital visual display 4404 with at least 100 pixels. The top-left embodiment demonstrates how the display shows target 4404A and current orientation 4404B. The target 4404A is always located in the center of the display, but the location of current orientation 4404B depends on the spatial orientation of the device compared to the desired orientation. The next three embodiments show how tilting the device results in the current orientation 4404B moving closer to and then into the circle of target orientation 4404A.

FIG. 45 shows a device with circular digital visual display 4504 and finger groove 4502. This finger receiving location 4502 has a second side surface that is only connected to the bottom surface along a part of its perimeter.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112(f) is not invoked.

What is claimed is:

1. A surgical device for guiding insertion of a needle along a desired three-dimensional trajectory from a surgical entry site to a surgical destination site, the surgical device comprising:
a housing;
a needle attachment element;
a spatial orientation indicator on an external surface of the housing;
an inertial measurement unit (IMU) inside the housing; and
an electronic communication element that electronically transmits inertial measurement data (IMD) from the IMU to a controller configured to determine the relationships between the device's current pitch and yaw and the pitch and yaw of the desired trajectory by comparing an initial yaw, an initial pitch, the IMD, and the desired three-dimensional trajectory.

2. The device of claim 1, wherein the needle attachment element is a bottom needle attachment element (BNAE) located at a bottom surface of the housing.

3. The device of claim 2, wherein the BNAE comprises a conical exterior surface.

4. The device of claim 2, wherein the BNAE comprises a cylindrical section and internal screw threads on an internal surface of the cylindrical section.

5. The device of claim 2, wherein the BNAE is a spring plunger BNAE comprising:
a body comprising a flange;
a cavity in the body;
a plunger; and
a spring,
wherein the spring is positioned between the body and the plunger,
wherein a first section of the plunger is positioned between the spring and the cavity,
wherein a second section of the plunger is positioned between the spring and the flange.

6. The device of claim 2, wherein the BNAE is a pinching BNAE comprising:
a body;
a cavity in the body;
a pinching element; and
a rotatable object that is partially located at an outer surface of the body,
wherein the pinching element is positioned between the body and the cavity,
wherein rotating the rotatable object causes the pinching element to move inwards towards the cavity.

7. The device of claim 1, wherein the needle attachment element is an inner surface of a hole in the housing.

8. The device of claim 2, wherein the hole has a circular cross-section.

9. The device of any one of claims 7-8, wherein the geometric center of the device is located in the hole, wherein the geometric center of the device is the arithmetic mean of all points on the housing.

10. The device of any one of claims 7-9, further comprising two or more clamps that can be moved to reduce the size of the hole of the housing.

11. The device of any one of claims 1-10, wherein the housing comprises a finger receiving location that is a second side surface that:
is connected to the first side surface along the entire bottom perimeter of the first side surface,
is connected to the bottom surface along the entire perimeter of the bottom surface, and has a larger horizontal cross section at its top than at its bottom.

12. The device of claim 11, wherein the second side surface is a curved surface that is concave out.

13. The device of claim 11 or 12, wherein the second side surface has a height ranging from 5 mm to 40 mm.

14. The device of any one of claims 11-13, wherein the first side surface and second side surface each comprise circular cross sections.

15. The device of any one of claims 11-14, wherein the second side surface has circular cross sections that are coaxial with a circular cross section of the side surface.

16. The device of any one of claims 11-15, wherein the second side surface has circular cross sections that decrease in diameter when moving from the top of the second side surface to the bottom of the second side surface.

17. The device of any one of claims 1-10, wherein the housing comprises a finger receiving location that is a second surface that:
is connected to the first side surface along a part of the perimeter of the first side surface, is connected to the bottom surface along a part of the perimeter of the bottom surface, and
has a larger horizontal cross section at its top than at its bottom.

18. The device of claim 16, wherein the second side surface is planar.

19. The device of claim 16, wherein the second side surface is a curved surface that is concave out.

20. The device of any one of claims 1-18, wherein the inertial measurement unit comprises a gyroscope, an accelerometer, or a combination thereof.

21. The device of claim 19, wherein the inertial measurement unit comprises a microelectromechanical system (MEMS) gyroscope.

22. The device of any one of claims 1-20, wherein the spatial orientation indicator comprises a marking.

23. The device of claim 21, wherein the marking comprises two line segments that intersect each other at a perpendicular angle.

24. The device of any one of claims 1-22, wherein the spatial orientation indicator comprises a protrusion.

25. The device of any one of claims 1-23, wherein the spatial orientation indicator is dimensioned for alignment with a grid of lights from a medical imaging device.

26. The device of claim 24, wherein the medical imaging device is selected from the group consisting of a computerized tomography (CT) scanner, a positron emission tomography (PET) scanner, and a magnetic resonance imaging (MRI) scanner.

27. The device of any one of claims 1-25, wherein the controller is located inside the housing and the electronic communication element comprises an electronic communication wire located entirely inside the housing.

28. The device of any one of claims 1-25, wherein the controller is located outside the housing, wherein the electronic communication element comprises a wireless electronic transmitter or an electronic communication wire comprising a section inside the housing and a section outside the housing.

29. The device of any one of claims 1-27, wherein the housing comprises one or more exterior indentations.

30. The device of claim 28, wherein the housing comprises two, three, or four exterior indentations that each independently have a depth ranging from 5 mm to 4 cm.

31. The device of any one of claims 1-29, further comprising two directional markings that are located on an exterior of the housing and each have a different color than surrounding regions of the exterior of the housing.

32. A system for guiding surgical insertion of a needle along a desired three-dimensional trajectory from a surgical entry site to a surgical destination site, the system comprising:
a surgical device of any one of claims 1-31; and
one or more device selected from the group consisting of:
the controller;
a display that receives and visually indicates the relationships between the current pitch and yaw of the device and the pitch and yaw of the desired trajectory;
the needle; and
a spacer comprising an opening, wherein the spacer has outer dimensions allowing a section of the spacer to be inserted into a hole of the housing.

33. The system of claim 32, wherein the system comprises the controller.

34. The system of claim 33, wherein the controller is located inside the housing.

35. The system of claim 33, wherein the controller is located outside the housing.

36. The system of any one of claims 32-35, wherein the system comprises the display.

37. The system of claim 36, wherein the display is attached to an external surface of the housing.

38. The system of claim 37, wherein the display is attached to a top, external surface of the housing.

39. The system of claim 36, wherein the display is separate from the housing.

40. The system of any one of claims 36-39, wherein the display comprises a digital visual display having at least 100 pixels located at the top surface of the device.

41. The system of claim 40, wherein the digital visual display has at least 400 pixels.

42. The system of claim 40 or 41, wherein the digital visual display is a multicolor digital visual display.

43. The system of any one of claims 40-42, wherein the digital visual display is configured to:
show a target orientation marker at the center of the digital visual display, show a current orientation marker on the display based on the relationship between the current pitch and yaw and the desired pitch and yaw.

44. wherein the digital visual display is configured to show the current orientation marker in a different color based on its position relative to the target orientation marker.

45. The system of any one of claims 40-44, wherein the digital visual display is flat and circular.

46. The system of any one of claims 36-39, wherein the display indicates numerical differences between the current pitch and yaw and the desired pitch and yaw.

47. The system of any one of claims 32-46, wherein the system comprises the needle.

48. The system of claim 47, wherein a section of the needle is positioned within the hole, and wherein frictional forces between the needle and the housing or the spacer prevent relative motion between the needle and housing due to gravity.

49. The system of any one of claims 32-48, wherein the system comprises the spacer.

50. The system of claim 49, wherein the hole has a circular cross-section and an inner diameter that is larger than the outer diameter of a circular cross-section of the spacer.

51. The system of any one of claims 49-50, wherein the system comprises a second spacer comprising a second opening, wherein the second spacer has outer dimensions allowing a section of the second spacer to be inserted into the hole of the housing, wherein the second opening has a diameter that is different from a diameter of the first opening.

52. A controller for using a surgical device to guide insertion of a needle along a desired three-dimensional trajectory from a surgical entry site to a surgical destination site,
wherein the surgical device is a surgical device of any one of claims 1-31;
wherein the controller is configured to:
determine a desired pitch and a desired yaw based on the desired three-dimensional trajectory; and
receive notice that the spatial orientation indicator is aligned with an initial reference and therefore the device has an initial pitch and an initial yaw;
receive the inertial measurement data (IMD) from the electronic communication element;
determine the device's current pitch and current yaw based on the initial pitch, the initial yaw, and the inertial measurement data;
transmit the relationships between the device's current pitch and yaw to the desired pitch and yaw to a display.

53. The controller of claim 52, wherein transmitting the relationships comprises transmitting a numerical value for offset of the pitch and a numerical value for offset of the yaw, wherein each offset is the difference between the desired value and the current value.

54. The controller of claim 52, wherein the transmitting the relationships comprises transmitting instructions that a particular colored light should be illuminated.

55. The controller of claim 52, wherein transmitting the relationships comprises transmitting instructions for which pixels of a digital visual display of 100 pixels or more should be illuminated.

56. The controller of claim 55, wherein transmitting the relationships comprises transmitting instructions for which pixels of a digital visual display of 100 pixels or more should be: (i) illuminated and, (ii) the color of illumination if illuminated.

57. A non-transitory computer readable storage medium with computer executable instructions stored thereon executed by a processor to perform a method of using a surgical device to guide insertion of a needle along a desired three-dimensional trajectory from a surgical entry site to a surgical destination site, the method comprising:
determine a desired pitch and a desired yaw based on the desired three-dimensional trajectory; and
receive notice that the spatial orientation indicator is aligned with an initial reference and therefore the device has an initial pitch and an initial yaw;
receive the inertial measurement data (IMD) from the electronic communication element;
determine the device's current pitch and current yaw based on the initial pitch, the initial yaw, and the inertial measurement data;
transmit the relationships between the device's current pitch and yaw to the desired pitch and yaw to a display.

58. A method for using a surgical device to guide insertion of a needle along a desired three-dimensional trajectory from a surgical entry site to a surgical destination site, the method comprising:
selecting the desired three-dimensional trajectory;
providing a system comprising:
a surgical device of any one of claims 1-31;
the controller;
the display that receives and visually indicates the relationships between the current pitch and yaw of the device and the pitch and yaw of the desired trajectory;
the needle;
orienting the device such that the spatial orientation indicator is aligned with an initial reference;
notifying the controller that the spatial orientation indicator is aligned with an initial reference, thereby causing the controller to determine that the device has an initial pitch and an initial yaw, and thereby also causing the controller to monitor the IMD and transmit the relationships between the device's current pitch and yaw to the desired pitch and yaw to the display;
moving the device to the surgical entry site;
observing the display and adjusting the pitch and yaw of the device such that the actual trajectory is more closely aligned with the desired trajectory;
if a section of the needle is not already positioned within the hole, moving the needle such that a section of the needle is positioned within the hole; and
moving the needle such that a distal end of the needle is inserted into the surgical entry site along the actual trajectory to a first depth.

59. The method of claim 57, wherein selecting the desired three-dimensional trajectory comprises:
recording a plurality of two-dimensional (2D) images of the surgical region with a medical imaging device;
generating a three-dimensional (3D) representation of the surgical region from the plurality of 2D images;
using the 3D representation to choose the desired trajectory from a surgical entry site to a surgical destination site.

60. The method of claim 59, wherein using the 3D representation to choose the desired trajectory is performed by a user.

61. The method of claim 59, wherein using the 3D representation to choose the desired trajectory is performed by a software algorithm.

62. The method of any one of claims 58-61, wherein using the 3D representation to choose the desired trajectory comprises avoiding anatomic structures.

63. The method of any one of claims 58-62, wherein the display is observed while simultaneously moving the needle to the first depth.

64. The method of any one of claims 58-62, wherein the display is not observed while moving the needle to the first depth.

65. The method of any one of claims 58-64, further comprising recording an image of the inserted needle at the first depth.

66. The method of claim 65, wherein the recorded image is a CT scan, a PET scan, or an MRI scan.

67. The method of any one of claims 58-66, further comprising:
selecting a first updated desired trajectory from the proximal end of the needle at the first depth to the surgical destination site;
adjusting the pitch and yaw of the device according to the display such that the first updated actual trajectory is more closely aligned with the first updated desired trajectory;
moving the needle such that the needle is inserted to a second depth into the surgical region along the first updated actual trajectory.

68. The method of any one of claims 58-66, further comprising removing a tissue sample from the surgical destination site with the needle.

69. The method of any one of claims 58-68, further comprising administering an active pharmaceutical ingredient (API) through the needle to the surgical destination site.

70. The method of any one of claims 58-69, further comprising ablating tissue at the surgical destination site with an ablation module inserted through the needle.

71. A kit for guiding insertion of a needle along a desired three-dimensional trajectory from a surgical entry site to a surgical destination site, the kit comprising:
a surgical device of any one of claims 1-31;
packaging containing the surgical device.

72. The kit of claim 71, further comprising the display.

73. The kit of any one of claims 71-72, further comprising the controller.

74. The kit of any one of claims 71-73, further comprising the needle.

75. The kit of any one of claims 71-74, further comprising a spacer comprising an opening, wherein the spacer has outer dimensions allowing a section of the spacer to be inserted into a hole of the housing.

76. The kit of claim 75, further comprising a second spacer comprising a second opening, wherein the second spacer has outer dimensions allowing a section of the second spacer to be inserted into the hole of the housing, wherein the second opening has a diameter that is different from a diameter of the first opening.

77. A surgical device for guiding insertion of a needle along a fixed three-dimensional orientation, the device comprising:
a principle axis defining an upwards direction and a downwards direction,
a first horizontal axis defining a left direction and a right direction,
a second horizontal axis defining a forward direction and a backwards direction,
(A) a base comprising a base hole;
(B) an upper housing comprising an upper hole,
wherein the upper housing is located at least partially within the base hole,
wherein the principle axis passes through the base hole and the upper hole,
wherein the upper housing can rotate around the principle axis and thereby rotate relative to the base,
(C) a track comprising a first rail and a second rail that are each parallel to the first horizontal axis,
wherein the first rail is located within the upper hole and comprises a first end and a second end that are each attached to the upper housing,
wherein the second rail is located within the upper hole and comprises a first end and a second end that are each attached to the upper housing,
(D) a needle receiver comprising a needle hole,
wherein the needle receiver is positioned between the first rail and the second rail of the track,
wherein the first and second rails limit the movement of the needle receiver in the upwards, downwards, forwards, and backwards directions,
wherein the first and second rails allow movement of the needle receiver parallel to the rails in the left and right directions,
wherein the needle receiver can rotate around the second horizontal axis and thereby rotate relative to the track,
(E) a needle aligner comprising a first roller and a second roller that are both connected to the upper housing,
wherein the first roller comprises a first cylindrical section configured to rotate around a first roller axis,
wherein the second roller comprises a second cylindrical section configured to rotate around a second roller axis,
wherein the first roller axis and second roller axis are both parallel to the first horizontal axis and therefore remain parallel to the first and second rails even if the upper housing is rotated relative to the base,
(F) wherein a horizontal plane of open space exists that is located: (i) between the first and second cylindrical sections of the rollers, (ii) directly above the space between the first rail and the second rail, (iii) directly above the upper hole, and (iv) directly above the base hole,
wherein the horizontal plane of open space is also referred to as the roller-roller space.

78. The device of claim 77, wherein the upper housing is connected indirectly to the base.

79. The device of any one of claims 77-78, wherein:
the device further comprises ball bearings and a middle housing comprising a middle hole,
the middle housing is located at least partially within the base hole,
the upper housing is located at least partially within the middle hole,
the ball bearings are positioned circumferentially around the upper housing and between the upper housing and the middle housing, thereby allowing the upper housing to rotate around the principle axis and relative to the middle housing.

80. The device of any one of claims 77-79, wherein the device further comprises a cover plate, wherein the first and second rollers are attached to the cover plate and the cover plate is attached to the upper housing.

81. A method of surgically inserting a needle along a fixed three-dimensional orientation into a surgical region of a patient, the method comprising:
providing a surgical device of any one of claims 77-80;
fixing the base of the surgical device to a skin surface of the patient;
providing the needle, wherein a diameter at a middle section of the needle is less than a distance between the first roller and the second roller at the roller-roller space;
moving the needle receiver in a left or right direction along the track to a particular position;
rotating the needle receiver around the second horizontal axis to a particular orientation;
moving a distal end of the needle into the roller-roller space and then down past the roller-roller space, thereby causing the middle section of the needle to make and maintain contact with both the first roller and the second roller, wherein further motion of the needle causes the first and second rollers to rotate;
moving the distal end of the needle into the needle hole,
moving the distal end of the needle downwards and out of the needle hole; and
moving the distal end of the needle into the surgical region of the patient.

82. The method of claim 81, wherein the needle receiver is rotated such that the needle hole is aligned with the principle axis.

83. The method of any one of claims 81-82, wherein the needle receiver is rotated such that the needle hole is angled relative to the principle axis.

84. The method of any one of claims 81-83, further comprising rotating the upper housing relative to the base,
thereby causing rotation of the first and second rollers and the first and second rails relative to the base,
thereby causing rotation of the needle relative to the base and therefore also causing movement of the distal end of the needle relative to the skin of the patient,
wherein rotating the upper housing relative to the base is performed after moving the distal end of the needle into the needle hole but before moving the distal end of the needle into the surgical region of the patient.

85. The method of any one of claims 81-84, wherein the method further comprises:
measuring the rotation of the first roller and optionally the second roller during each moving step;
determining the depth of needle insertion based on the measured rotation of the first and optionally second rollers;
after the determining, moving the distal end of the needle a particular distance based on the determined depth.

86. The method of any one of claims 81-85, moving the needle receiver in a left or right direction along the track after the distal end of the needle is inserted into the needle hole but before the distal end is moved into the surgical region of the patient.

87. A method of surgically inserting a needle along a fixed three-dimensional orientation into a surgical region of a patient, the method comprising:
providing a surgical device comprising:
a principle axis defining an upwards direction and a downwards direction,
a first horizontal axis defining a left direction and a right direction,
a second horizontal axis defining a forward direction and a backwards direction,
(A) a base comprising a base hole;
(B) an upper housing comprising an upper hole,
wherein the upper housing is located at least partially within the base hole,
wherein the principle axis passes through the base hole and the upper hole,
wherein the upper housing can rotate around the principle axis and thereby rotate relative to the base,
(C) a track comprising a first rail and a second rail that are each parallel to the first horizontal axis,
wherein the first rail is located within the upper hole and comprises a first end and a second end that are each attached to the upper housing,
wherein the second rail is located within the upper hole and comprises a first end and a second end that are each attached to the upper housing,
(D) a needle receiver comprising a needle hole,
wherein the needle receiver is positioned between the first rail and the second rail of the track,
wherein the first and second rails limit the movement of the needle receiver in the upwards, downwards, forwards, and backwards directions,
wherein the first and second rails allow movement of the needle receiver parallel to the rails in the left and right directions,
wherein the needle receiver can rotate around the second horizontal axis and thereby rotate relative to the track,
(E) a needle aligner comprising a first roller and a second roller that are both connected to the upper housing,
wherein the first roller comprises a first cylindrical section configured to rotate around a first roller axis,
wherein the second roller comprises a second cylindrical section configured to rotate around a second roller axis,
wherein the first roller axis and second roller axis are both parallel to the first horizontal axis and therefore remain parallel to the first and second rails even if the upper housing is rotated relative to the base,
(F) wherein a horizontal plane of open space exists that is located: (i) between the first and second cylindrical sections of the rollers, (ii) directly above the space between the first rail and the second rail, (iii) directly above the upper hole, and (iv) directly above the base hole,
wherein the horizontal plane of open space is also referred to as the roller-roller space.
fixing the base of the surgical device to a skin surface of the patient;
providing the needle, wherein a diameter at a middle section of the needle is less than a distance between the first roller and the second roller at the roller-roller space;
moving the needle receiver in a left or right direction along the track to a particular position;
rotating the needle receiver around the second horizontal axis to a particular orientation;
moving a distal end of the needle into the roller-roller space and then down past the roller-roller space, thereby causing the middle section of the needle to make and maintain contact with both the first roller and the second roller, wherein further motion of the needle causes the first and second rollers to rotate;
moving the distal end of the needle into the needle hole,
moving the distal end of the needle downwards and out of the needle hole; and
moving the distal end of the needle into the surgical region of the patient.
